SEQUENCE LISTING (9 sequences)

SEQ ID NO: 1 - DNA, 20 bp, Homo sapiens
  primer_bind (1)..(20): sense primer for human PAI-1
tggaacaaggatgagatcag

SEQ ID NO: 2 - DNA, 20 bp, Homo sapiens
  primer_bind (1)..(20): antisense primer for human PAI-1
ccgttgaagtagagggcatt

SEQ ID NO: 3 - DNA, 20 bp, Homo sapiens
  primer_bind (1)..(20): sense primer for human alpha-SMA
ctgttccagccatccttcat

SEQ ID NO: 4 - DNA, 20 bp, Homo sapiens
  primer_bind (1)..(20): antisense primer for human alpha-SMA
ccgtgatctccttctgcatt

SEQ ID NO: 5 - DNA, 21 bp, Homo sapiens
  primer_bind (1)..(21): sense primer for glyceraldehyde-3-phosphate dehydrogenase
gaccccttcattgacctcaac

SEQ ID NO: 6 - DNA, 20 bp, Homo sapiens
  primer_bind (1)..(20): antisense primer for glyceraldehyde-3-phosphate dehydrogenase
cttctccatggtggtgaaga

SEQ ID NO: 7 - DNA, 21 bp, Artificial Sequence
  primer
cgggatgttcacaaccgaatt

SEQ ID NO: 8 - DNA, 21 bp, Artificial Sequence
  primer
aacaagaccttcgactcttcc

SEQ ID NO: 9 - DNA, 21 bp, Artificial Sequence
  primer
gttctccgaacgtgtcacgtt

(19) United States
(12) Patent Application Publication
(10) Pub. No.: US 2011/0319405 A1
(43) Pub. Date: Dec. 29, 2011

(54) Treatment and prevention of diffuse parenchymal lung disease by selective active-site mTOR inhibitors

(75) Inventors: Glenn D. Rosen, Los Altos, CA (US); Wen-Teh Chang, San Jose, CA (US)

(73) Assignee: The Board of Trustees of the Leland Stanford Junior University, Palo Alto, CA (US)

(21) Appl. No.: 13/171,441

(22) Filed: Jun. 28, 2011

Related U.S. Application Data
(60) Provisional application No. 61/398,622, filed on Jun. 28, 2010.
(60) Provisional application No. 61/459,317, filed on Dec. 8, 2010.

(51) Int. Cl. A61K 31/519; A61P 29/00; A61P 11/00; A61P 17/00; A61K 31/496; A61K 31/5377

(52) U.S. Cl. 514/232.5; 514/262.1; 514/253.03; 514/234.2

(57) ABSTRACT

Embodiments are related to new uses for selective active-site mTOR inhibitors in treating or preventing pulmonary fibrosis in diffuse parenchymal lung disease (DPLD) patients, such as a DPLD of environmental cause, a collagen vascular disease (e.g., scleroderma and rheumatoid arthritis), an idiopathic interstitial pneumonia (e.g., idiopathic pulmonary fibrosis and nonspecific interstitial pneumonia), and sarcoidosis.

### RELATED APPLICATION

This application claims priority and other benefits from U.S. Provisional Patent Applications Ser. No. 61/398,622, filed Jun. 28, 2010, entitled “Inhibition of Pulmonary Fibrosis by Dual mTOR Inhibitors”, and Ser. No. 61/459,317, filed Dec. 8, 2010, entitled “Treatment and Prevention of Pulmonary Fibrosis by Selective Active-Site mTOR Inhibitors”. Their entire content is specifically incorporated herein by reference.

### TECHNICAL FIELD OF THE INVENTION

The present invention relates to the field of bioaffecting and body-treating methods of using active-site mTOR inhibitors.

### BACKGROUND

Diffuse parenchymal lung diseases (DPLDs), also known as interstitial lung diseases, are devastating diseases of often unknown cause, usually with poor prognosis and limited treatment options.

Idiopathic pulmonary fibrosis (IPF) is a chronic and particularly progressive form of diffuse parenchymal lung disease that is characterized by an accumulation of scar tissue in the lung's interstitium and for which currently no effective pharmacological treatment is available in the United States, except for pirfenidone which is approved in several countries and continents outside of the United States, such as in Europe, Australia, India, Southkorea and Japan. Idiopathic pulmonary fibrosis occurs primarily in individuals over the age of 50, who have a median survival time of 4-5 years, before they succumb to respiratory failure.

There is an urgent and unmet need to identify agents and methods for the effective treatment of idiopathic pulmonary fibrosis and other fibrotic diseases.

### SUMMARY

The present invention provides methods for treating diffuse parenchymal lung diseases and for blocking their pathogenesis.

A first embodiment is a method for treating or preventing pulmonary fibrosis in an individual suffering from a diffuse parenchymal lung disease and administering an amount of a pharmaceutical composition comprising a selective active-site mTOR inhibitor to said individual effective to treat or prevent pulmonary fibrosis in said individual. Another embodiment is a method for determining treatment efficacy of an individual's treatment with an active-site mTOR inhibitor by assessing said individual's lung function over the course of the treatment.

A further embodiment is a method for inhibiting expression of α-SMA or collagen or fibronectin in a pulmonary fibroblast comprising administering an amount of a selective active-site mTOR inhibitor to a pulmonary fibroblast in an individual suffering from a diffuse parenchymal lung disease effective to inhibit expression of α-SMA or collagen or fibronectin in said pulmonary fibroblast of said individual.

In further embodiments said diffuse parenchymal lung disease is a diffuse parenchymal lung disease of environmental cause or a collagen vascular disease, such as scleroderma or rheumatoid arthritis, or an idiopathic interstitial pneumonia or sarcoidosis.

In other embodiments, said idiopathic interstitial pneumonia is idiopathic pulmonary fibrosis (IPF) or nonspecific interstitial pneumonia (NSIP). In certain embodiments said selective active-site mTOR inhibitor is a pyrazolopyrimidine.

In other embodiments, said selective active-site mTOR inhibitor is defined as being selective on the basis of inhibiting other PI3Ks only at ≧ about 10-fold higher concentrations.

In other embodiments said selective active-site mTOR inhibitor is defined as being an ATP-competitive inhibitor of mTOR on the basis of one or more assays, as reported in Thoreen C. C. et al., J. Biol. Chem., 2009, 284, 8023-8032; Feldman M. E. et al., PLoS Biol., 2009, 7, e38; García-Martínez J. M. et al., Biochem. J., 2009, 421, 29-42; Yu K. et al., Cancer Res., 2009, 69, 6232-6240.

In some embodiments said selective active-site mTOR inhibitor is Torin 1, PP242, Ku-0063794, or WAY-600.

The above summary is not intended to include all features and aspects of the present invention nor does it imply that the invention must include all features and aspects discussed in this summary.

INCORPORATION BY REFERENCE

All publications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

Figure A. Diffuse parenchymal lung diseases (DPLDs) encompass a collection of disorders. Am J Respir Crit Care Med., 2002, 165, 277-304.

Figure B. The diagnostic method in diffuse pulmonary lung diseases (DPLDs) is based on clinical, radiological, and pathological criteria. Am J Respir Crit Care Med., 2002, 165, 277-304.

Figure C. mTORC1 and mTORC2 are distinguished by different compositions, substrates, and functions. J Biol Chem., 2010, 285, 14071-7.

Figure D. In vitro IC₅₀ Values for PP242 and PP30 determined in the presence of 10 μM ATP. PLoS Biol., 2009, 7, e38.

Figure E. Schematic of signaling through SPARC in IPF fibroblasts. J Biol Chem., 2010, 285, 8196-206.

Figure F. Working model of dual mTOR signaling in pulmonary fibrogenesis. Cf. Wang, S. et al., Am J Physiol Renal Physiol., 2010, 298, F142-9. Following TGF-β stimulation in pulmonary fibroblasts, the noncanonical and canonical (through Smad2/Smad3) TGF-β pathways closely collaborate. The pathways induce transition of fibroblasts into myo-fibroblasts and the expression in these fibroblasts and myo-fibroblasts of stromal proteins, including α-smooth muscle actin (α-SMA) and multiple extracellular matrix proteins, such as collagen and fibronectin. According to Wang et al., supra, the noncanonical pathway also induces fibroblast proliferation through the Akt-mTORC1-S6K branch.

FIG. 1 illustrates that active site or dual mTOR inhibitors, PP242 or Ku-0063794 (which block both mTORC1 and mTORC2) are more effective than rapamycin (which primarily blocks mTORC1) in suppressing expression of scar-forming extracellular matrix proteins, type I collagen and secreted protein acidic and rich in cysteine (SPARC) from fibroblasts, which are derived from patients with idiopathic pulmonary fibrosis (IPF). Active-site mTOR inhibitors PP242 and Ku-0063794, but not rapamycin, block phosphorylation of Akt at Ser473, a target of mTORC2.

FIG. 2 illustrates that transforming growth factor β (TGF-f3), a major mediator of fibrosis in IPF, activates Akt (Serine 473 and Threonine 308) in normal lung fibroblasts, both of which are blocked by active-site mTOR inhibitors PP242 or Ku-0063794, but not by rapamycin. PP242 or Ku-0063794 is more effective than rapamycin in blocking induction by TGF-β of type I collagen, α-smooth muscle actin (α-SMA) and fibronectin, all proteins made by activated fibroblasts, which promote scar formation in IPF. Also, PP242 proved more effective than rapamycin or Ku-0063794 in blocking induction by TGF-β of plasminogen activator inhibitor 1 (PAI-1). As seen in FIG. 2D, none of these inhibitors affect the Smad pathway, which is the major TGF-β-regulated pathway.

FIG. 3 illustrates that turning off expression of Rictor, a component of the mTORC2 pathway, which when inhibited blocks mTORC2 but not mTORC1, shows similar repressive effects on these pro-fibrotic proteins to PP242 or Ku-0063794 in IPF fibroblasts and TGF-β-stimulated control fibroblasts. It is illustrated that a specific Akt inhibitor shows similar effects but not identical, suggesting that activation of Akt by mTORC2 mediates expression of some pro-fibrotic proteins by IPF fibroblasts and control TGF-β-stimulated fibroblasts.

FIG. 4 illustrates that PP242 significantly inhibited fibrosis in the bleomycin murine model of lung injury and fibrosis, which is a well-accepted animal model of lung injury and fibrosis. PP242 significantly inhibits expression of important matrix regulated proteins, which are upregulated by bleomycin and known targets of TGF-β, such as SPARC, metastasin (MTS-1/S100A4), and matrix metalloproteinase 7 (MMP-7). Quantitation of hydroxyproline content at day 14 in the right lung of saline treated mice, mice treated with Bleomycin plus vehicle and mice treated with Bleomycin plus pp242 one day prior to the administration of Bleomycin. Results are the average of three independent experiments with mean±SD.

FIG. 5 illustrates that PP242 or repression of Rictor inhibited migration of control or IPF fibroblasts.

### DEFINITIONS

The practice of the present invention may employ conventional techniques of chemistry, molecular biology, recombinant DNA, genetics, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002); Paul Singleton and Diana Sainsbury, Dictionary of Microbiology and Molecular Biology (3d ed. revised, John Wiley & Sons, Chichester, England, 2006). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “drug” means, as used herein and as stated in the Federal Food, Drug, and Cosmetic Act, an article intended for use in the diagnosis, cure, mitigation, treatment, or prevention of disease in man or other animal.

The term “biologic” means, as used herein and as defined by the Food and Drug Administration, a subset of drugs that are distinguished by the biological manufacturing process. These definitions serve to differentiate a drug substance from a drug product.

The term “pharmaceutical composition”, as used herein, refers to a mixture of at least one active-site mTOR inhibitor with chemical components such as diluents or carriers that do not cause unacceptable adverse side effects and that do not prevent the active-site mTOR inhibitor(s) from exerting a therapeutic effect. A pharmaceutical composition serves to facilitate the administration of the active-site mTOR inhibitor(s).

Routes of administration of active-site mTOR inhibitors or pharmaceutical compositions containing active-site mTOR inhibitors may include, but are not limited to, oral or topical administration and intramuscular, subcutaneous, intravenous or intraperitoneal injections. The active-site mTOR inhibitors or pharmaceutical compositions containing active-site mTOR inhibitors may also be administered locally via an injection or in a targeted delivery system.

The term “active-site mTOR inhibitors”, as used herein, relates to biologically active, recombinant, isolated peptides and proteins, including their biologically active fragments, peptidomimetics and small molecules that are capable of inhibiting mTOR complex 1 (mTORC1) and mTOR complex 2 (mTORC2). In comparison, rapamycin and rapamycin analogs exert their action through inhibition of only mTORC1; mTORC2 does not seem to be affected by rapamycin and rapamycin analogs.

The term “therapeutic effect”, as used herein, refers to a consequence of treatment with an active-site mTOR inhibitor or active-site mTOR inhibitors that might intend either to bring mitigation or treatment of an injury that already occurred or to prevent an injury before it occurs. Efficacy studies may be carried out to provide evidence of an active-site mTOR inhibitor's ability in mitigation, treatment or prevention of a diffuse parenchymal lung disease. Mitigation, treatment, or prevention does not necessarily mean 100% efficacy. Rather, mitigation, treatment, or prevention may mean some level of efficacy, anywhere from 1% to 100%.

A therapeutic effect may include, directly or indirectly, the reduction of pulmonary interstitial fibrosis and the reduction of pulmonary interstitial inflammation. A therapeutic effect may also include, directly or indirectly, the arrest, reduction, or elimination of pulmonary interstitial fibrogenesis or extracellular matrix deposition. Furthermore, a therapeutic effect may include, directly or indirectly, the prevention or reduction of the expression of stromal genes constitutively expressed in idiopathic pulmonary fibrosis fibroblasts including, but not limited to fibronectin (FN), type-1-collagen, alpha smooth muscle actin (α-SMA), secreted protein acidic and rich in cysteine (SPARC) and plasminogen activator inhibitor 1 (PAI-1). Furthermore, a therapeutic effect may also include, directly or indirectly, the prevention or reduction of the expression of transforming growth factor beta (TGF-β).

The term “therapeutically effective amount” of an active-site mTOR inhibitor is an amount that is sufficient to provide a therapeutic effect in a mammal, including a human. Naturally, dosage levels of the particular active-site mTOR inhibitor employed to provide a therapeutically effective amount may vary in dependence of the type of injury, the age, the weight, the gender, the medical condition of the mammal/human, the severity of the condition, the route of administration, and the particular active-site mTOR inhibitor employed. Therapeutically effective amounts of an active-site mTOR inhibitor, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The therapeutic efficacy of treating an individual with an active-site mTOR inhibitor can be determined by repeatedly assessing said individual's lung function over the course of the treatment. Spirometry is the most common method to assess lung function, specifically measuring the amount (volume) and speed (flow) of air that can be inhaled and exhaled. An increase in lung function over time, in comparison to prior lung function tests, indicates therapeutic efficacy of treatment with an active-site mTOR inhibitor. Similarly, unchanged lung function over time, thus indicating an arrest in worsening of lung function, in comparison to prior lung function tests, can also indicate therapeutic efficacy of treatment with an active-site mTOR inhibitor. Accordingly, a decrease in lung function over time, in comparison to prior lung function tests, can indicate a lack of therapeutic efficacy of treatment with an active-site mTOR inhibitor.

### DETAILED DESCRIPTION

The present invention provides methods for treating diffuse parenchymal lung diseases and for blocking their pathogenesis.

### Diffuse Parenchymal Lung Diseases

Diffuse Parenchymal Lung Disease. Referring to Figure A, diffuse parenchymal lung diseases (DPLDs), also known as interstitial lung diseases, consist of disorders of known causes (collagen vascular disease, environmental or drug related) as well as disorders of unknown cause. The latter include idiopathic interstitial pneumonias (IIPs), granulomatous lung disorders (e.g., sarcoidosis), and other forms of interstitial lung disease (ILD) including lymphangioleiomyomatosis (LAM), pulmonary Langerhans' cell histiocytosis/histiocytosis X (HX), and eosinophilic pneumonia. The most important distinction among the idiopathic interstitial pneumonias is that between idiopathic pulmonary fibrosis and the other interstitial pneumonias (IPs), which include nonspecific interstitial pneumonia (a provisional term), desquamative interstitial pneumonia, respiratory bronchiolitis-associated interstitial lung disease, acute interstitial pneumonia, cryptogenic organizing pneumonia, and lymphocytic interstitial pneumonia.

Referring to Figure B, the diagnostic method in diffuse pulmonary lung diseases (DPLDs) begins with a clinical evaluation that includes a history, physical examination, chest radiograph, and lung function tests. On the basis of this information, the patients may be divided into two groups: cases that do not represent idiopathic interstitial pneumonia (IIP), owing to recognition of associated conditions or underlying exposures, and cases that could represent IIP. Patients in the latter category typically receive a high-resolution computerized tomography (HRCT) scan. This generally results in four categories of patients: (1) those with distinctive features that allow for a confident diagnosis of idiopathic pulmonary fibrosis (IPF)/usual interstitial pneumonia (UIP) in the appropriate clinical setting, (2) those with atypical clinical or CT features for IPF, (3) those with features diagnostic of another DPLD such as pulmonary Langerhans' cell histiocytosis (PLCH), and (4) those with suspected other forms of DPLD. Although many patients will go directly to surgical lung biopsy, some patients may undergo transbronchial biopsy (TBBx) or bronchoalveolar lavage (BAL). If these findings are nondiagnostic a surgical lung biopsy may be necessary to separate the various IIPs from non-IIP DPLD.

In general, most diffuse pulmonary lung disease is characterized by particular microscopic patterns of inflammation and fibrosis. Fibrosis is characterized by an increased amount and abnormal structure of the connective tissue. Inflammation is characterized by excessive inflammatory cells.

Idiopathic Interstitial Pneumonia. Referring, again, to Figure A, the idiopathic interstitial pneumonias (IIPs) are a group of diffuse parenchymal lung diseases (DPLDs). The IIPs are a heterogeneous group of nonneoplastic disorders resulting from damage to the lung parenchyma by varying patterns of inflammation and fibrosis. The interstitium includes the space between the epithelial and endothelial basement membranes and it is the primary site of injury in the IIPs. However, these disorders frequently affect not only the interstitium, but also the airspaces, peripheral airways, and vessels along with their respective epithelial and endothelial linings.

Idiopathic indicates unknown cause and interstitial pneumonia refers to involvement of the lung parenchyma by varying combinations of fibrosis and inflammation. The idiopathic interstitial pneumonias include the entities of idiopathic pulmonary fibrosis (IPF), nonspecific interstitial pneumonia (NSIP), cryptogenic organizing pneumonia (COP), acute interstitial pneumonia (AIP), respiratory bronchiolitis-associated interstitial lung disease (RB-ILD), desquamative interstitial pneumonia (DIP), and lymphocytic interstitial pneumonia (LIP).

The current clinico-pathologic classification of the idiopathic interstitial pneumonias (IIPs) is summarized in Table 1. See Am J Respir Crit Care Med., 2002, 165, 277-304; Strollo D. C., Am J Respir Cell Mol Biol., 2003, 29(3 Suppl), S10-18.

TABLE 1
Classification of Idiopathic Interstitial Pneumonias

| Diagnosis | Radiology | Distribution | Pathology |
|---|---|---|---|
| IPF/UIP | Fibrosis, | Basilar, peripheral | Temporal |
| | honeycombing | | heterogeneity, |
| | | | fibroblastic foci, |
| | | | fibrotic and normal |
| | | | lung, microscopic |
| | | | honeycombing |
| NSIP | Ground glass | Basilar, peripheral | Diffuse interstitial |
| | opacity +/− | | inflammation +/− |
| | fibrosis | | fibrosis |
| COP | Ground glass | Patchy upper lungs, | Granulation tissue |
| | opacity, nodules, | small airways, | plugs in alveolar |
| | consolidation | alveolar | ducts and alveoli |
| AIP | Ground glass | Diffuse, random | Hyaline |
| | opacity, | | membranes, |
| | consolidation | | immature |
| | | | fibroblasts in |
| | | | alveolar spaces and |
| | | | interstitium to |
| | | | variable degree |
| RB-ILD | Bronchiectasis, | Upper lungs, | Respiratory |
| | ground glass | bronchocentric | bronchiolitis |
| | opacity | | surrounded by |
| | | | macrophages in |
| | | | alveoli |
| DIP | Ground glass | Basilar, peripheral, | Alveolar |
| | opacity, | alveolar | macarophages in |
| | consolidation | | air spaces diffusely |
| | | | in the biopsy |
| LIP | Ground glass | Patchy | Lymphoid |
| | opacity, nodules, | | hyperplasia |
| | cysts | | |

Idiopathic Pulmonary Fibrosis. The terms usual interstitial pneumonia (UIP) and idiopathic pulmonary fibrosis (IPF) have become more narrowly defined since they were originally proposed several decades ago. The relationship between historically defined IPF, or cryptogenic fibrosing alveolitis (CFA), and UIP has been described in Am J Respir Crit Care Med., 2000, 161, 646-664. According to the current definition, IPF is a distinctive type of chronic fibrosing interstitial pneumonia of unknown cause limited to the lungs and associated with a surgical lung biopsy showing a histologic pattern of UIP. Am J Respir Crit Care Med., 2000, 161, 646-664.

A dysregulated wound healing response to lung epithelial injury, which leads to progressive interstitial fibrosis, is a hallmark of the disease. The fibrotic scar is generated, in large part, by activated fibroblasts, which are sometimes referred to as myofibroblasts, based on their expression of alpha smooth muscle actin (α-SMA). Lung fibroblasts transdifferentiate into myofibroblasts in response to TGF-β, which is released largely by injured type I or type II alveolar epithelial cells and activated macrophages in fibroblastic foci in IPF lung. These myofibroblasts produce type I and type III collagen, which compose the fibrotic interstitial scar, and other mesenchymal proteins such as secreted protein acidic and rich in cysteine (SPARC), plasminogen activator inhibitor 1 (PAI-1), and fibronectin (FN).

In the presence of a surgical biopsy showing a UIP pattern the diagnosis of IPF requires (1) exclusion of other known causes of interstitial lung disease including drug toxicities, environmental exposures, and collagen vascular diseases, (2) characteristic abnormalities on conventional chest radiographs or high-resolution computed tomography (HRCT) scans, and (3) abnormal pulmonary function studies showing restriction (reduced total lung capacity [TLC], or reduced vital capacity [VC] with a normal or increased FEV₁/FVC ratio) and/or impaired gas exchange [increased P(A-a)O₂ (alveolar-arterial pressure difference for O₂), decreased Pa_{O2} with rest or exercise, or decreased DL_{CO} (diffusing capacity of the lung for CO)].

The new American Thoracic Society (ATS)/European Respiratory Society (ERS)/Japanese Respiratory Society (JRS)/Latin American Thoracic Society (ALAT) guidelines for the diagnosis and management of IPF were published in the Am J Respir Crit Care Med, 2011, 183, 788-824. However, lung biopsy may occasionally not be definitive. This may arise when there is histologic heterogeneity in different lobes of the lung in IPF. So after correlating all the clinical, radiological, and pathological information, the final diagnosis may still be IPF in a patient with typical clinical-radiological IPF, even though a lung biopsy shows a fibrosing NSIP pattern.

Clinical Features. Onset of symptoms is usually gradual, with dyspnea the most prominent and disabling symptom. A nonproductive cough is usual and may be paroxysmal. It is often refractory to antitussive agents. The patient's age at onset is usually greater than 50 yr and IPF is slightly more common in males. Constitutional symptoms are unusual. Digital clubbing develops in 25 to 50% of patients, and Velcro-type fine end-inspiratory crackles that are initially confined to the basal areas are found on chest auscultation. These progress gradually to involve the entire lung. Features of right heart failure and peripheral edema develop only in the late stages. Most patients exhibit a restrictive pattern of ventilatory defect with a decrease in DL_{CO} and low resting Pa_{O2}, which falls on exercise. Pulmonary function or chest radiographs may be normal or near normal in the early phase of IPF. In smokers and ex-smokers with IPF, coexistent chronic obstructive pulmonary disease may result in relatively higher lung volumes compared with never-smoking patients with IPF.

In most patients, symptoms have been present for more than 6 month before presentation. The clinical course is invariably one of gradual deterioration. Median length of survival from time of diagnosis varies between 4-5 yrs. Occasionally, periods of rapid decline are recognized. These may represent accelerated disease, intercurrent viral infection with the development of organizing pneumonia, or diffuse alveolar damage. Improvement in lung physiology and radiologic abnormalities is rare.

Bronchoalveolar lavage fluid contains an excess of neutrophils, the proportions of which correspond to the extent of reticular change on HRCT. There may also be a mild or moderate increase in the percentage of eosinophils. BAL cell counts, although correlating with severity of disease, do not predict prognosis. When eosinophils represent more than 20% of the count, consideration should be given to an eosinophilic lung disease. Lymphocytosis is not a feature of UIP, and counts above 15% should alert to an alternative diagnosis such as NSIP, COP, hypersensitivity pneumonitis, sarcoidosis, or other granulomatous lung disease.

Radiologic Features. The commonest chest radiographic abnormality in patients with IPF is peripheral reticular opacity, most marked at the bases, and often associated with honeycombing and lower lobe volume loss. In patients with associated upper lobe emphysema, the radiographic lung volumes may be normal or even increased. Chest radiographs may occasionally be normal in patients with IPF.

UIP is characterized on CT by the presence of reticular opacities, often associated with traction bronchiectasis. Honeycombing is common. Ground glass opacities are uncommon, but ground glass attenuation, which is associated with areas of fibrosis, can often be seen. Architectural distortion, reflecting lung fibrosis, is often prominent. Lobar volume loss is seen with more advanced fibrosis. The distribution of UIP on CT is characteristically basal and peripheral, although often patchy. On serial scans in treated patients, honeycomb cysts usually enlarge slowly over time.

Reticular abnormality on CT correlates with fibrosis on histopathologic examination. Honeycombing on CT correlates with honeycombing on biopsy. The CT pattern of UIP due to IPF can be indistinguishable from that found in UIP due to asbestosis and to collagen vascular disease. The presence of pleural plaques or diffuse pleural thickening helps to distinguish asbestosis from IPF. Patients with chronic hypersensitivity pneumonitis, or with end-stage sarcoidosis, may uncommonly develop a CT pattern similar to that of UIP. Hypersensitivity pneumonitis should be considered if poorly defined fine micronodules are seen, or if there is sparing of the lung bases. Sarcoidosis should be suspected if the cysts are large, or if peribronchovascular nodules are present.

Histologic Features. The key histologic features of the UIP pattern are architectural destruction, fibrosis often with honeycombing, scattered fibroblastic foci, patchy distribution and involvement of the periphery of the acinus or lobule. It has a heterogeneous appearance at low magnification, with alternating areas of normal lung, interstitial inflammation, fibrosis, and honeycomb change. The histological changes affect the peripheral subpleural parenchyma most severely. Interstitial inflammation is usually mild to moderate, patchy, and consists of an alveolar septal infiltrate of lymphocytes, plasma cells, and histiocytes associated with hyperplasia of Type II pneumocytes. The fibrotic zones show temporal heterogeneity with dense acellular collagen and scattered fibroblastic foci. Areas of honeycomb change are composed of cystic fibrotic airspaces, which are frequently lined by bronchiolar epithelium and filled with mucin. Smooth muscle hyperplasia is commonly seen in areas of fibrosis and honeycomb change. Areas of relatively normal lung should be present in surgical biopsy specimens in order to exclude the presence of active lesions of other interstitial disorders. Otherwise the UIP pattern may be difficult to recognize and a pathologist may only be able to diagnose “severe fibrosis with honeycomb change.” In some patients with a UIP pattern on lung biopsy, specimens from a second or third lobe of lung may not fulfill the histologic criteria for UIP and suggest other patterns such as NSIP. However, in such a setting the default pathologic diagnosis is UIP.

Patients who are biopsied during an accelerated phase of their illness may show a combination of UIP pattern and a variety of acute lesions. These include infection, prominent organizing pneumonia, diffuse alveolar damage, and capillaritis. If no cause can be determined this may represent “accelerated decline of IPF” or acute exacerbation of IPF. A pattern of interstitial inflammation and fibrosis nearly indistinguishable from that seen in UIP can occur in patients with collagen vascular diseases, certain drug-induced lung diseases, chronic hypersensitivity pneumonitis, asbestosis, and familial IPF. There is no single histologic finding that has shown a consistent correlation with treatment response or prognosis in IPF.

The differential diagnosis of the IIPs must be approached in two ways: histologically and clinically. In interpreting lung biopsies, the pathologist must address the differential diagnosis on the basis of the histologic pattern. A search should be made for histologic clues to a potential cause such as asbestos bodies, infectious agents, or other exogenous agents. The clinician must address most of the etiologic possibilities and in most cases ultimately determines whether the process is idiopathic.

The histologic differential diagnosis of the UIP pattern includes the histologic patterns of the other IIPs including fibrosing NSIP, DIP, organizing pneumonia, chronic hypersensitivity pneumonitis, and diffuse alveolar damage. With the narrowing of the histologic definition of the UIP pattern there are only a few clinical conditions that may cause an identical histologic pattern. Lesions that can present histological features similar but not identical to UIP include asbestosis, collagen vascular disease, the fibrosing phase of hypersensitivity pneumonitis, radiation pneumonitis, and Hermansky-Pudlak syndrome.

Collagen Vascular Disease. Collagen vascular or connective tissue disorders are a group of autoimmune diseases in which antibodies attack the body's own organs and systems. Among the many targets of these auto-antibodies is connective tissue, which is the supporting structure for all of the body's cells. An important component of connective tissue is the protein, collagen. Abnormalities in blood vessel structure and function are also typical, accounting for the term “collagen vascular diseases,” which is often used interchangeably with connective tissue disorders. These disorders typically feature inflammation and fibrosis in several organs and tissues. The joints are frequently involved, particularly in the most common of these conditions, rheumatoid arthritis. Thus, rheumatology is the primary medical subspecialty involved in the diagnosis and care of these patients. Lung involvement may complicate the course of most of these conditions and sometimes can dominate the clinical picture.

The frequency and type of lung involvement in connective tissue disorders varies based on the underlying disease. Of all the connective tissue disorders, scleroderma, also known as systemic sclerosis, is most likely to affect the lungs. Pulmonary fibrosis occurs in over two thirds of scleroderma patients, but pulmonary fibrosis also occurs in rheumatoid arthritis, mixed connective tissue disease, systemic lupus erythematosus, Sjögren's syndrome, and polymyositis/dermatomyositis.

Scleroderma. Scleroderma, also known as systemic sclerosis, is a rheumatic and autoimmune disease characterized by skin thickening and effects on the vascular system, lung, kidney, gastrointestinal tract, and heart. The criteria for the classification of scleroderma are published as Arthritis Rheum 1980, 23, 581-90.

Rheumatoid Arthritis. Rheumatoid arthritis (RA) is a rheumatic and autoimmune disease characterized by inflammation in the joints. The 1987 American College of Rheumatology criteria are used in the clinical diagnosis of rheumatoid arthritis, and to define rheumatoid arthritis in epidemiologic studies. Persons must meet four of seven ACR criteria; these criteria are based on clinical observation (e.g., number of joints affected), laboratory tests (e.g., positive rheumatoid factor), and radiographic examination (e.g., X-rays evidence of joint erosion). Arnett F C et al., Arthritis Rheum 1988, 31, 315-324.

Historically, pharmacologic treatment of RA has traditionally followed the pyramid approach. That is, treatment starts with corticosteroids/non-steroidal anti-inflammatory drugs, then progresses to disease-modifying anti-rheumatic drugs and finally to biologic response modifiers, if persons are non-responsive to the previous drugs. Arthritis Rheum 2002, 46, 328-346.

Mixed Connective Tissue Disease. Mixed connective tissue disease (MCTD) is an uncommon, specifically defined, overlap syndrome characterized by clinical features of systemic lupus erythematosus, scleroderma, and polymyositis/dermatomyositis with very high titers of circulating antinuclear antibody to a ribonucleoprotein antigen. The criteria for MCTD are published as Alarcon-Segovia D, Cardiel M H. J Rheumatol 1989, 16, 328-34.

Systemic Lupus Erythematosus. Systemic lupus erythematosus (SLE) is a rheumatic and autoimmune disease characterized by fatigue, body aches, skin rash, neurologic complaints, and effects on the kidney, lung, and heart. The American College of Rheumatology (ACR) 1982 Revised Criteria for SLE are published as Tan E M et al., Arthritis Rheum 1982, 25, 1271-7. The ACR 1982 Revised Criteria for SLE Update is published as Hochberg M C. Arthritis Rheum 1997, 40, 1725.

Sjögren's Syndrome. Sjögren's syndrome (SS) is a rheumatic and autoimmune disease characterized by dry eyes and dry mouth, and effects on the joints. The American College of Rheumatology (ACR) Criteria for SS are published as Fox R I et al., Arthritis Rheum 1986, 29, 577-85.

Polymyositis/Dermatomyositis. Polymyositis/Dermatomyositis (PM/DM) is a rheumatic and autoimmune disease characterized by muscle weakness, difficulty swallowing, rash around eyes, and effects on the heart and lung. The criteria for PM/DM are published as Bohan A, Peter J B. N Engl J Med 1975, 292, 344-7 (first of two parts) and Bohan A, Peter J B. N Engl J Med 1975, 292, 403-7 (second of two parts); or Bohan A et al., Medicine (Baltimore) 1977, 56, 255-86.

### Fibrogenesis as Dysregulated Wound Healing Response to Lung Epithelial Injury

IPF is a progressive deadly disease that is characterized by gradual fibrosis of the supporting framework, the interstitium, of the lungs. Currently, there is no approved pharmacological treatment available in the United States and lung transplantation is usually the only option to prolong survival. Although the exact pathogenesis of IPF is unknown, a dysregulated wound healing response to lung epithelial injury, which leads to progressive interstitial fibrosis, is a hallmark of the disease. Hereby, transforming growth factor-β (TGF-β) appears to play a central role in mediating induction of stromal genes, which sets off fibrogenesis.

The fibrotic scar is generated, in large part, by activated fibroblasts, which are sometimes referred to as myofibroblasts, based on their expression of alpha smooth muscle action (α-SMA). Lung fibroblasts transdifferentiate into myofibroblasts in response to TGF-β, which is released largely by injured type I or type II alveolar epithelial cells and activated macrophages in fibroblastic foci in lungs affected by idiopathic pulmonary fibrosis. The myofibroblasts produce type I and type III collagen, which compose the fibrotic interstitial scar, and other mesenchymal proteins such as secreted protein acidic and rich in cystein (SPARC), plasminogen activator inhibitor 1 (PAI-1) and fibronectin (FN).

The Mammalian Target of Rapamycin (mTOR)

mTOR and Signaling. Referring to Figure C, the mammalian target of rapamycin (mTOR) is a 289-kDa serine/threonine protein kinase, a member of the PI3K-related kinase (PIKK) family (Sarbassov, D. D., et al., Phosphorylation and regulation of Akt/PKB by the rictor-mTOR complex. Science, 2005. 307(5712): p. 1098-101). The mTOR pathway integrates both extracellular and intracellular signals and acts as a central regulator of cell metabolism, growth, proliferation and survival (Proud, C. G., mTORC1 signalling and mRNA translation. Biochem Soc Trans, 2009. 37(Pt 1): p. 227-31).

To date, two mTOR complexes have been characterized: mTOR complex 1 (mTORC1), a rapamycin-sensitive complex, and mTOR complex 2 (mTORC2), a rapamycin-insensitive complex (Hay, N. and N. Sonenberg, Upstream and downstream of mTOR. Genes Dev, 2004. 18(16): p. 1926-45). In mammalian cells, mTOR resides in two physically and functionally distinct signaling complexes, mTORC1 and mTORC2 (Huang, S. K., et al., Prostaglandin E2 induces fibroblast apoptosis by modulating multiple survival pathways. FASEB J, 2009). mTORC1 consists of at least five components: (i) mTOR, the catalytic subunit of the complex; (ii) Raptor; (iii) mLS8; (iv) PRAS40; and (v) Deptor. mTORC1 phosphorylates the ribosomal S6K1 (protein S6 kinase 1) and 4E-BP1 (eukaryotic translation initiation factor eIF4E binding protein 1) proteins, which regulate growth and protein synthesis, respectively (Jacinto, E., et al., Mammalian TOR complex 2 controls the actin cytoskeleton and is rapamycin insensitive. Nat Cell Biol, 2004. 6(11): p. 1122-8). Rapamycin and rapalogs inhibit phosphorylation of S6K1 and 4E-BP1 through inhibition of mTORC1 (Serini, G., et al., The fibronectin domain ED-A is crucial for myofibroblastic phenotype induction by transforming growth factor-betal. J Cell Biol, 1998. 142(3): p. 873-81).

mTORC2 is a rapamycin-insensitive complex, consisting of six different known proteins: (i) mTOR; (ii) Rictor; (iii) mSIN1; (iv) Protor-1; (v) mLST8; and (vi) Deptor. Rictor and mSIN1 mutually stabilize each other, thus establishing the structural foundation of the complex (Jacinto, E., et al., Mammalian TOR complex 2 controls the actin cytoskeleton and is rapamycin insensitive. Nat Cell Biol, 2004. 6(11): p. 1122-8). Deptor negatively regulates mTORC2 activity, and mLST8 is also necessary for the stability and activity of the complex. mTORC2 has been shown to mediate the phosphorylation of Akt on Ser 473, leading to activation of the Akt pathway, which initiates several cancer-related cellular responses, including increased cell growth and proliferation, a shift to glycolytic metabolism, and increased cell migration (Wu, L. and R. Derynck, Essential role of TGF-beta signaling in glucose-induced cell hypertrophy. Dev Cell, 2009. 17(1): p. 35-48). There is also evidence suggesting that mTORC2 phosphorylates PKCα and regulates the organization of the actin cytoskeleton (Bhagwat, S. V. and A. P. Crew, Novel inhibitors of mTORC1 and mTORC2. Curr Opin Investig Drugs. 11(6): p. 638-45). PI3K also mediates a PDK1-dependent activation of Akt at Thr 308 in response to growth factors, such as insulin growth factor type 1 (IGF-1) (Sarbassov, D. D., et al., Phosphorylation and regulation of Akt/PKB by the rictor-mTOR complex. Science, 2005. 307(5712): p. 1098-101).

The mRNA sequence of human mTOR is published as NCBI Reference Sequence NM_{—}004958.3. The protein sequence of human mTOR is published as NCBI Reference Sequence NP_{—}004949.1 The mTORC1 and mTORC2 complexes contain shared and distinct partner proteins and control a myriad of cellular processes in response to diverse environmental cues. Recent studies have indirectly implicated mTORC2 as a target of TGF-β. In a study by White et al, for example, the authors showed that TGF-β activates Akt (Ser473) in fetal lung fibroblasts, which led to induction of fibronectin (FN) expression and increased generation of the alternatively spliced extra type III domain A (EDA) fibronectin variant (White, E. S., et al., Control of fibroblast fibronectin expression and alternative splicing via the PI3K/Akt/mTOR pathway. Exp Cell Res, 2010. 316(16): p. 2644-53; Chang, W. T., et al., Triptolide and chemotherapy cooperate in tumor cell apoptosis. A role for the p53 pathway. J Biol Chem, 2001. 276(3): p. 2221-7). The EDA spliced variant of FN is expressed by fibroblasts in fibroblastic foci in IPF lung and regulates TGF-β-mediated conversion of fibroblasts into myofibroblasts (Ashcroft, T., J. M. Simpson, and V. Timbrell, Simple method of estimating severity of pulmonary fibrosis on a numerical scale. J Clin Pathol, 1988. 41(4): p. 467-70). The authors further showed that rapamycin inhibited expression of FN and EDA-FN, without affecting Akt (Ser473) phosphorylation, and thereby implicated mTORC1 as the key regulator of FN and EDA-FN expression by fibroblasts exposed to TGF-β.

Another recent study showed that glucose regulates cell hypertrophy in fibroblasts and epithelial cells through TGF-β-mediated activation of mTOR (mTORC1 and mTORC2) (Wu, L. and R. Derynck, Essential role of TGF-beta signaling in glucose-induced cell hypertrophy. Dev Cell, 2009. 17(1): p. 35-48). The discovery of ATP-competitive mTORC1/2 inhibitors was recently reported by several research groups, but a selective mTORC2 inhibitor has yet to be developed. A few dual active-site mTOR inhibitors have progressed to clinical trials for cancer (Bhagwat, S. V. and A. P. Crew, Novel inhibitors of mTORC1 and mTORC2. Curr Opin Investig Drugs. 11(6): p. 638-45). The PP242, Ku-0063794, and Wye-354 compounds are commercially available (Chemdea, Ridgewood, N.J.). Because Akt is constitutively phosphorylated at Ser473 in IPF fibroblasts and Akt is activated by TGF-β, mTORC2 possibly mediates both constitutive Akt phosphorylation (Ser473) in IPF fibroblasts and its activation by TGF-β. Blocking Akt activation with an active-site mTOR inhibitor might succeed in inhibiting the expression of mesenchymal genes, which are downstream of Akt.

### Rapamycin

The bacterial macrolide-derived rapamycin interacts with the cellular protein FKBP12, and this complex directly binds to the mTOR FKBP12-rapamycin-binding (FRB) domain to allosterically inhibit mTORC1. Although the mechanism by which rapamycin inhibits mTORC1 remains incompletely defined, rapamycin weakens the interaction between mTOR and raptor (regulatory associated protein of mTOR), an mTORC1 regulatory partner, and reduces mTORC1 intrinsic kinase activity. Chronic high-dose rapamycin inhibits mTORC2 signaling in certain cell types by impeding mTORC2 assembly, however.

mTORC1 and mTORC2 contain shared and unique partners. Each complex contains mTOR, mLST8/GβL, and deptor. mLST8/GβL binds the mTOR kinase domain in both complexes but appears more critical for mTORC2 assembly and signaling. Deptor functions as an inhibitor of both complexes. Other partner proteins distinguish the two complexes. mTORC1 contains exclusively raptor and PRAS40. Raptor functions as a scaffolding protein that links the mTOR kinase with mTORC1 substrates to promote mTORC1 signaling. PRAS40 functions in a regulatory capacity. In contrast, mTORC2 contains exclusively rictor (rapamycin-insensitive companion of mTOR), mSin1, and PRR5/protor. Rictor and mSin1 promote mTORC2 assembly and signaling; the function of PRR5/protor remains obscure.

mTORC1 senses and integrates diverse extra- and intracellular signals to promote anabolic and inhibit catabolic cellular processes. Growth factors and nutrients (e.g. amino acids, energy) promote mTORC1-dependent protein synthesis, cell growth (increase in cell mass/size), cell proliferation, and cell metabolism. Conversely, insufficient levels of these factors, or signals of cell stress, blunt mTORC1 action to maintain cellular biosynthetic rates appropriate for suboptimal cellular conditions. Reduced mTORC1 signaling also promotes macroautophagy, a degradative process that enhances cell survival in the face of decreased nutrient availability via the breakdown of cell constituents into amino acids and other small molecules. TORC1 in yeast and mammals also promotes “ribosome biogenesis,” a process whereby mTORC1 increases the transcription of ribosomal RNAs and proteins to augment cellular protein biosynthetic capacity.

Raptor binds directly to mTOR signaling (TOS) motifs on downstream targets, including S6K1 (ribosomal S6 protein kinase 1) and 4EBP1 (eukaryotic initiation factor (eIF) 4E-binding protein 1) (as well as PRAS40 and Hif1α), thus linking them to the mTOR kinase. The TOS motif is required for mTOR/raptor-mediated phosphorylation of S6K1 on its hydrophobic motif (HM) site (Thr389) and 4EBP1 on multiple sites (Thr37/46, Thr70, Ser65). Raptor mutation within its raptor N-terminal conserved domain abrogates 4EBP1 binding and mTORC1-mediated 4EBP1 phosphorylation in vitro while retaining mTOR interaction, thus underscoring the importance of the raptor-4EBP1 interaction for mTORC1 signaling. As the avidity of the raptor-mTOR interaction increases during nutrient and growth factor insufficiency (when mTORC1 signaling is low), raptor may possess opposing cell condition-dependent functions in mTORC1 regulation.

The serine-threonine protein kinase Akt (also known as protein kinase B) represents the first identified substrate of mTORC2. Akt promotes cell proliferation, cell survival, and cell migration and controls various metabolic processes. Full activation of Akt in response to growth factor-mediated PI3K signaling requires dual phosphorylation on its activation loop site (Thr308) by PDK1 and HM site (Ser473) by mTORC2. mTORC2 also phosphorylates the HM sites on SGK1 (Ser422) and protein kinase Cα (PKCα; Ser657). As the mTORC1 substrate S6K1 and the mTORC2 substrates Akt, PKCα, and SGK1 represent AGC kinases, an emerging theme in mTOR signaling is that mTORC1 and mTORC2 phosphorylate members of the AGC kinase family.

Active-site mTOR Inhibitors

The molecules Torin 1, PP242 (a pyrazolopyrimidine), Ku-0063794, and WAY-600 are representative members of the class of selective and ATP-competitive inhibitors of mTOR. Thoreen C. C. et al., J. Biol. Chem., 2009, 284, 8023-8032; Feldman M. E. et al., PLoS Biol., 2009, 7, e38; García-Martínez J. M. et al., Biochem. J., 2009, 421, 29-42; Yu K. et al., Cancer Res., 2009, 69, 6232-6240.

Unlike rapamycin, these molecules inhibit both mTORC1 and mTORC2, and, unlike PI3K family inhibitors such as LY294002, these molecules inhibit mTOR with selectivity relative to PI3Ks and protein kinases. To distinguish these molecules from the allosteric mTORC1 inhibitor rapamycin, we are calling them active-site mTOR inhibitors for TOR kinase (catalytic) domain inhibitors.

PI3Ks catalyze the synthesis of the phosphatidylinositol (PI) second messengers PI(3)P, PI(3,4)P₂, and PI(3,4,5)P₃ (PIP₃). In the appropriate cellular context, these three lipids control diverse physiological processes including cell growth, survival, differentiation, and chemotaxis. The PI3K family comprises 15 kinases with distinct substrate specificities, expression patterns, and modes of regulation. The class I PI3Ks (p110α, p110β, p110δ, and p110γ) are activated by tyrosine kinases or G protein-coupled receptors to generate PIP₃, which engages downstream effectors such as the Akt/PDK1 pathway, the Tec family kinases, and the Rho family GTPases. The class II and III PI3Ks play a key role in intracellular trafficking through the synthesis of PI(3)P and PI(3,4)P₂. The PI3K-related kinases (PIKKs) are protein kinases that control cell growth (mTORC1) or monitor genomic integrity (ATM, ATR, DNA-PK, and hSmg-1).

Referring to Figure D, PP242 and PP30 inhibit mTOR in vitro with half-maximal inhibitory concentrations (IC₅₀ values) of 8 nM and 80 nM, respectively. As expected for active-site inhibitors, PP242 and PP30 inhibit mTOR in both mTORC1 and mTORC2. Referring to Figure D, both compounds are selective within the PI3K family, inhibiting other PI3Ks only at ≧ about 10-fold higher concentrations. Apsel B. et al., Nat Chem Biol, 2008, 4, 691-699 tested PP242 against 219 purified protein kinases at a concentration 100-fold higher than its mTOR IC₅₀ value revealing selectivity with respect to the protein kinome; most protein kinases were unaffected by this drug, and only four—PKC-alpha, PKC-beta, RET, and JAK2—were inhibited more than 80%. Feldman M. E. et al., PLoS Biol., 2009, 7, e38 determined IC₅₀ values for PP242 against these kinases in vitro using purified proteins. In these assays, and referring to FIGURE FIGURE D, PP242 was relatively inactive against PKC-beta, RET, or JAK2 but inhibited PKC-alpha with an in vitro IC₅₀ of 50 nM. These data indicate that PP242 is a selective active-site inhibitor of mTOR.

### Pirfenidone

Pirfenidone, currently approved outside of the United States for the treatment of idiopathic pulmonary fibrosis, is an orally active synthetic antifibrotic agent that inhibits fibroblast, epidermal, platelet-derived, and transforming beta-1 growth factors (according to the National Cancer Institute's dictionary), thereby slowing down progression of the fibrosis, while stabilizing lung function. Pirfenidone also inhibits DNA synthesis and the production of mRNA for collagen types I and III, resulting in a reduction in radiation-induced fibrosis. Pirfenidone does not appear to act on any of the mTOR complexes.

### Utility of the Present Invention

The inventors of the present invention identified pathways existent in IPF fibroblasts and TGF-β-stimulated normal fibroblasts, which mediate expression of stromal genes and found that the mTOR/Akt pathway is activated in IPF fibroblasts and in TGF-β-stimulated normal lung fibroblasts. Furthermore, the mTOR pathway, both mTORC1 and mTORC2, was found to regulate stromal gene expression in both fibroblast populations.

In comparison to rapamycin, which inhibits mTORC1, active-site mTOR inhibitors, which inhibit both mTORC1 and mTORC2, proved to be more effective in suppressing basal expression of pro-fibrotic proteins in IPF fibroblasts and the same group of proteins induced by TGF-β from control lung fibroblasts.

Also, specifically blocking the mTOR pathway by turning off Rictor recapitulated the suppressive results seen with PP242 or Ku-0063794.

Moreover, in the murine bleomycin lung fibrosis model, active-site mTOR inhibitor PP242 significantly inhibited lung fibrosis and expression of stromal genes. These data confirm active-site mTOR inhibitors may be effective in the treatment and prevention of idiopathic pulmonary fibrosis and in other diffuse parenchymal lung diseases. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### EXPERIMENTAL PROCEDURES

The following methods and materials were used in the examples that are described further below.

Cell Culture and Reagents. Lung fibroblasts were isolated from patients undergoing surgical biopsy for the diagnosis of interstitial lung disease or lung transplant for IPF, and non-neoplastic tissue was obtained from patients undergoing surgical lung cancer resection, as described previously (Chang W. T. et al., J Biol Chem., 2001, 276, 2221-7). Fibroblasts were cultured in Dulbecco's modified Eagle's medium (DMEM) supplemented with 10% fetal bovine serum (FBS) at 5% CO₂ at 37° C., and were used for studies for up to 5 passages after the initial preparation. For each experiment, cells were plated in culture vessels in DMEM complete medium and cultured until 70-80% in confluence, unless otherwise indicated. Cells were subjected to starvation by washing cells twice with 1× phosphate-buffered saline (PBS), followed by adding DMEM/0.1% serum to each well and incubated for an additional 24 hours before further treatment with recombinant human TGF-β1 (2 ng/ml, Sigma, St. Louis, Mo.), recombinant human Wnt3a (25 ng/ml, R&D Systems, Minneapolis, Minn.), PP242 or Ku-0063794 (Chemdea, Ridgewood, N.J.).

Lung tissue was obtained from patients undergoing surgical biopsy for the diagnosis of interstitial lung disease or lung transplant for IPF, and non-neoplastic tissue was obtained from patients undergoing surgical lung cancer resection. The tissue was minced into small pieces with a scalpel and digested with type I collagenase (1 mg/ml; Invitrogen) and hyaluronidase (125 units/ml; Sigma) at 37° C. with agitation for 18 h in Dulbecco's modified Eagle's medium (DMEM; Invitrogen) supplemented with 10% fetal bovine serum. The dissociated tissues were incubated without shaking for 5 min at room temperature, followed by the separation of cell-enriched supernatant into a new tube. The cell fraction was centrifuged at 250×g for 5 min, and the pellet was then resuspended in DMEM with 10% fetal bovine serum. Epithelial cells did not, in general, survive more than one passage and were in large part eliminated through trypsinization. Surviving fibroblasts were cultured in DMEM supplemented with 10% fetal bovine serum at 5% CO₂ at 37 ° C. Each fibroblast culture was frozen at its earliest available passage and was used for studies for up to five passages. For each experiment, cells were plated in culture vessels in DMEM and cultured until 70-80% confluent, unless indicated otherwise. Cells were subjected to starvation by washing cells twice with 1× phosphate-buffered saline (PBS), followed by the addition of DMEM and 0.1% serum to each well and incubation for an additional 24 h. In the experiments for cell death induced by Glu-plasminogen (American Diagnostica Inc., Stamford, Conn.), phenol red-free DMEM was used.

Western Blot Analysis. Western blot analysis and band intensity quantitation were performed as described previously (Chang W. T. et al., J Biol Chem., 2001, 276, 2221-7). Briefly, the protein concentration was measured by Bradford assay (Bio-Rad) according to the manufacturer's instructions. An equal amount of protein was separated by SDS-PAGE and transferred to polyvinylidene difluoride membrane. Immunoblotting was performed using primary antibodies for α-SMA (American Research Products, Inc., Belmont, Mass.); SPARC (Biodesign International, Saco, Me.); cleaved caspase-3 (Asp¹⁷⁵), phospho-Akt (Ser⁴⁷³), phospho-GSK-3β (Ser⁹), and GSK-3β (Cell Signaling Technology, Danvers, Mass.); active β-catenin (8E7; Millipore, Billerica, Mass.); total β-catenin (BD Biosciences); PAI-1 (Santa Cruz Biotechnology, Santa Cruz, Calif.); and α-tubulin (used as a loading control; Sigma) overnight at 4° C., followed by incubation with the appropriate horseradish peroxidase-conjugated secondary antibody (GE Healthcare). The blot was visualized by enhanced chemiluminescence (GE Healthcare) and analyzed using a Kodak Image Station 4000R system (Carestream Health, Rochester, N.Y.).

To detect secreted PAI-1, the culture medium was first cleared by centrifugation, and proteins were precipitated in the presence of ammonium sulfate at 50% saturation overnight at 4° C. with gentle agitation. The excess salts were removed by dialysis against lysis buffer used for total lysate preparation, and protein quantitation was performed by the Bradford assay as described above. To suppress endogenous PI3K activity, 10 μm LY293002 or 1 μm wortmannin (Sigma) was used, and dimethyl sulfoxide (Sigma) was used as a control.

Immunohistochemistry staining of IPF lung tissue sections. Lung tissues isolated from patients undergoing surgical biopsy for the diagnosis of interstitial lung disease or lung transplant for IPF were fixed in 4% paraformaldehyde, dehydrated, paraffin embedded, and sectioned at 5-μm. To block endogenous peroxidase activity, sections were treated with 0.3% hydrogen peroxide in methanol for 20 min, preincubated with 5% goat serum, and treated with anti-α-SMA antibody (1:500), anti-type I collagen antibody (1:500), anti-SPARC antibody (1:500), or anti-phosphor-Akt antibody (1:500) for 1 h at 37° C. Then, the sections were incubated with a biotinylated goat secondary antibody, treated with avidin-biotin complex (Elite ABC kit, Vector Laboratories), and stained with diaminobenzidine tetrahydrochloride and hydrogen peroxide. Images were taken and processed using microscopy (Labophot-2, Nikon) equipped with a microscope digital camera system (Olympus).

Immunofluorescent Staining and Nuclear Localization of β-Catenin. The immunofluorescent staining and visualization of nuclear β-catenin were described previously (Chang W. T. et al., J Biol Chem., 2001, 276, 2221-7). Briefly, fibroblasts were grown on two-well chamber slides (Thermo Fisher Scientific, Rochester, N.Y.) in complete culture media until 50-60% confluence. Cells were subjected to starvation (0.1% serum) for 24 hours. After washing, fixing, and blocking, cells were then incubated with anti-active β-catenin (8E7, Millipore's Corporate, Billerica, Mass.) at 1:100 dilution in blocking solution for 16 hours at 4° C. The target proteins were visualized with FITC (fluorescein isothiocyanate)-conjugated secondary antibody (Calbiochem, San Diego, Calif.), and DAPI (4′,6-diamidino-2-phenylindole, Molecular Probes, Eugene, Oreg.) was used for nuclear counterstaining. Fluorescence images were taken and processed using microscopy (Labophot-2) equipped with Episcopic-fluorescence attachment (EFD-3, Nikon Instruments Inc., Melville, N.Y.).

Fibroblasts were grown on 2-well chamber slides (Thermo Fisher Scientific) in complete culture medium until 50-60% confluent. Cells were subjected to starvation (0.1% serum) for 48 h. After washing with 1×PBS, cells were treated with 100% methanol at −20 ° C. for 5 min. Cells were then blocked in 1×PBS and 3% normal goat serum (Sigma) for 30 min at room temperature, followed by incubation with anti-active β-catenin at 1:100 dilution in blocking solution for 16 h at 4° C. The target proteins were visualized with fluorescein isothiocyanate-conjugated secondary antibody (Calbiochem), and 4′,6-diamidino-2-phenylindole (Molecular Probes, Eugene, Oreg.) was used for nuclear counterstaining. Fluorescent images were taken and processed using a Labophot-2 microscope equipped with an episcopic fluorescence attachment (EFD-3; Nikon Instruments Inc., Melville, N.Y.). For the quantitation of nuclear localized β-catenin, at least total 100 cells were randomly selected from five fields of each stained sample to obtain the percentage of nuclear localization of β-catenin.

Tcf/LEF-driven luciferase reporter assay. Lentiviral-based constructs containing Tcf/LEF transcriptional response element (TOPFlash), or mutant (FOPFlash), were transduced into control lung fibroblasts. A green fluorescent protein (GFP)-coded lentiviral construct was co-transduced into cells as an equalizing reference. At 48 hours post infection, cells were placed into 24-well plates, starved, and treated in a triplicate manner with rapamycin, PP242, or Ku-0063794 plus TGF-β (2 ng/ml) or Wnt3a (25 ng/ml) for 24 hours. Reporter activity was determined in a multi-well plate reader by Luciferase Assay System (Promega). Luciferase activity was normalized for infection efficiency by measuring GFP activity from each well.

Nuclear Isolation, Staining, and Fluorescence-activated Cell Sorting. To isolate stable cell nuclei from fibroblasts for the staining of intranuclear β-catenin and for analyzing on a flow cytometer, a protocol disrupting cell membrane by detergent (Triton X-100) and maintaining nuclear membrane integrity by magnesium was adopted from the Flow Cytometry Core Laboratory at the NCI ETI Branch (home.ncifcrf.gov/ccr/flowcore/nuclei.pdf). Briefly, cells were collected by gentle scraping and washed twice with cold PBS. Cells were then resuspended in cold nuclear isolation buffer (320 mm sucrose, 5 mm MgCl₂, 10 mm HEPES, and 1% Triton X-100, pH 7.4) and allowed to incubate on ice for 10 min. Nuclear yield and integrity were confirmed by microscopic examination with trypan blue staining. We routinely observed >98% nuclear isolation efficiency (data not shown). Nuclei were pelleted by centrifugation at 2000×g and washed twice with nuclear wash buffer (320 mm sucrose, 5 mm MgCl₂, and 10 mm HEPES, pH 7.4). Isolated nuclei were then incubated overnight with anti-β-catenin antibody (5 μg/ml) or normal mouse IgG (Santa Cruz Biotechnology), followed by a 1-h incubation with fluorescein isothiocyanate-conjugated anti-mouse IgG (2 μg/ml) in nuclear wash buffer plus 1% bovine serum albumin and 0.1% sodium azide. All steps described above were done at 4° C. After extensive washing, nuclei were resuspended in 250 μl of nuclear wash buffer before flow cytometry analysis. Flow cytometry was performed on an Accuri C6 flow cytometer system (Accuri Cytometers, Inc., Ann Arbor, Mich.) using 488 nm excitation and standard fluorescein isothiocyanate emission optics with 10,000 events from each sample, and analysis was performed using FlowJo software (Tree Star Inc., Ashland, Oreg.).

Analysis of Gene Expression. RNA was extracted from fibroblasts using TRIzol (Invitrogen), and cDNA converted from 5 μg of total RNA was obtained using a SuperScript first-strand synthesis system for reverse transcription-PCR kit (Invitrogen). To control for genomic DNA contamination, additional RNA samples were processed without reverse transcriptase. The reverse transcription product equivalent to 25 ng of total RNA was then added to a real-time quantitative PCR (qPCR) using a Dynamo SYBR Green qPCR kit (Finnzymes, Espoo, Finland) according to the manufacturer's protocol. The following primers were used: human PAI-1, 5′-TGGAACAAGGATGAGATCAG-3′ (sense) and 5′-CCGTTGAAGTAGAGGGCATT-3′ (antisense); human α-SMA, 5′-CTGTTCCAGCCATCCTTCAT-3′ (sense) and 5′-CCGTGATCTCCTTCTGCATT-3′ (antisense); and glyceraldehyde-3-phosphate dehydrogenase, 5′-GACCCCTTCATTGACCTCAAC-3′ (sense) and 5′-CTTCTCCATGGTGGTGAAGA-3′ (antisense). The annealing and amplification temperature was 60° C. Real-time qPCR was performed in strip tubes using a StepOne PCR system (Applied Biosystems, Foster City, Calif.) according to the manufacturer's instructions. The specificity of amplified products was suggested by a melting curve resulting in only one peak.

This was further confirmed by agarose gel electrophoresis of the PCR products visualized under ethidium bromide/UV illumination. Target amplifications were compared with the reference amplifications (glyceraldehyde-3-phosphate dehydrogenase) in the same experiment for each reverse transcription product tested. All reactions were carried out in duplicate, and the threshold cycle (Cₜ) values were determined by automated threshold analysis with the StepOne software. The final results are presented as relative—fold change in target gene expression compared with the reference based on the comparative or ΔΔCₜ method. The efficiency of each primer pair was determined by the qPCR procedure from standard dilutions of cDNA (equivalent to 10 pg to 10 ng of total RNA in the reverse transcription reaction). To assess the effect of an inhibitor of the TGF-β1 receptor/ALK5 in suppressing PAI-1 expression, 10 μm SB431542 (Tocris Bioscience, Ellisville, Mo.) was added 1 h prior to the addition of human recombinant TGF-β1 at 2 ng/ml overnight.

Enzyme-linked Immunosorbent Assay for Secreted PAI-1. To determine the concentration of PAI-1 in the culture medium of human lung fibroblasts, cells were plated in a 24-well plate at 5×10⁵ cells/well overnight in DMEM and 10% fetal bovine serum. After serum deprivation for 24 h, the culture medium was collected and spun at 10,000×g for 5 min at 4° C. to remove any cell debris. The PAI-1 concentration of each supernatant was determined using a Quantikine human serpin E1/PAI-1 immunoassay kit (R&D Systems, Minneapolis, Minn.) according to the manufacturer's instructions.

Determination of TGF-β Bioactivity. The active TGF-β concentration in the cultured fibroblasts was assayed using the co-culture method with mink lung epithelial cells transfected with a truncated PAI-1 promoter fused to a firefly luciferase gene (referred to as MLEC-PAI-1-Lux cells; a kind gift from Dr. George Yang, Stanford University) (Abe M et al. (1994) Anal. Biochem. 216, 276-284). Fibroblasts were seeded in 96-well plates at 2.5×10⁴ cells/well in triplicates, along with MLEC-PAI-1-Lux cells at 1.5×10⁴ cells/well in low serum (1%) medium. The use of 1% serum instead of starvation medium with 0.1% serum was needed to support survival of the MLEC-PAI-1-Lux cells. A standard curve of active TGF-β1 was generated in MLEC-PAI-1-Lux cells with serial dilutions of human recombinant TGFβ1 (0-10 ng/ml; Sigma). After incubation for 24 h, the viability of cells was checked microscopically before washing twice with 1×PBS. Cells were then lysed in 1× passive lysis buffer at 50 μl/well (luciferase assay kit, Promega, Madison, Wis.) and incubated with agitation at room temperature for 20 min. 10 μl of cell lysate was analyzed for luciferase activity according to the manufacturer's instructions. Cells from fibroblast-only wells were used for the cell count. The mean values of luciferase activity from triplicates were then converted into concentrations of TGF-β in picograms/number of cells using a standard curve obtained with human recombinant TGF-β1, normalized with cell numbers. The concentration of total TGF-β1 in the culture medium for corresponding fibroblasts was determined using a Quantikine human TGF-β1 immunoassay kit (R&D Systems) according to the manufacturer's instructions.

Murine model of bleomycin-induced pulmonary fibrosis. Six-week-old male C57BL/6 mice weighing between 20 to 23 g were obtained from The Jackson Laboratory (Bar Harbor, Me.) and all care was in accordance with the National Institutes of Health Guide for Animal Welfare Act. Mice were administrated 0.1 ml of sterile saline (N=5) or bleomycin (Blenoxane, Novaplus, 1.5 U/kg of body weight; VHA Inc., Nippon Kayaku Co., Tokyo, Japan) at day 0 via intratracheal instillation under anesthesia (Krishna, G. et al., Am J Pathol. 2001, 158, 997-1004). Mice (N=5) received vehicle (15% PVP) or PP242 daily by oral route at 30 mg/kg of body weight starting the day prior to bleomycin treatment for six days a week. All mice were sacrificed on day 14 and the right lungs were first fixed by inflation with buffered 10% formalin solution, then incubated in the same solution for 24 hours. After paraffin-embedding, sections were processed, and stained with hematoxylin and eosin (H&E) for light microscopy. The left lungs were frozen in liquid nitrogen for subsequent analysis of hydroxyproline content.

Hydroxyproline assay to measure collagen content. To estimate the total amount of collagen deposited as an indicator of pulmonary fibrosis, the measurement of hydroxyproline content (Woessner, J. F., Jr., Arch Biochem Biophys., 1961, 93, 440-7) of whole lung was conducted for each sample according to the manufacture's instruction (Biocolor Ltd., UK). Briefly, Sirius red reagent was added to each lung homogenate (20 ml) and mixed for 30 min. The collagen-dye complex was precipitated by centrifugation at 16,000 g for 5 min, washed with ethanol, and dissolved in 0.5 M NaOH. The concentration of collagen in each sample was measured as absorbance at 540 nm and values extrapolated from a known standard curve as per manufacturer's instructions.

Histological Scoring of Fibrosis. The severity of fibrosis from each lung section was assessed by Ashcroft score (Ashcroft T. et al., J Clin Pathol, 1988, 41, 467-70). Briefly, the grade of lung fibrosis was scored on a scale from 0 to 8 by examining 30 randomly chosen fields per sample at a magnification of ×100. Criteria for grading lung fibrosis were as follows: grade 0, normal lung; grade 1, minimal fibrous thickening of alveolar or bronchiolar walls; grade 3, moderate thickening of walls without obvious damage to lung architecture; grade 5, increased fibrosis with definite damage to lung structure and formation of fibrous bands or small fibrous masses; grade 7, severe distortion of structure and large fibrous areas; grade 8, total fibrous obliteration of fields. If there was difficulty in deciding between two odd-numbered categories, the field would be given the intervening even-numbered grade. Scoring of the saline+vehicle and saline+PP242 groups both gave values of zero.

RNA Interference. We constructed a lentivirus-driven β-catenin small hairpin RNA (shRNA) expression plasmid from the pLKO.1 vector (22), targeting to β-catenin (5′-CGGGATGTTCACAACCGAATT-3′; pLKO.1-shβ-Cat), SPARC (5′-AACAAGACCTTCGACTCTTCC-3′; pLKO.1-shSPARC), or a scrambled sequence (5′-GTTCTCCGAACGTGTCACGTT-3′; pLKO.1-Scr). pLKO.1-shβ-Cat, pLKO.1-shSPARC, or pLKO.1-Scr was transduced into cells, followed by puromycin selection at 2 μg/ml for at least 48 h. The efficiency of shRNA knockdown of endogenous β-catenin or SPARC was assessed by Western blot analysis.

Quantitation of Cell Viability and Caspase-3 Activity Assay. Cell viability was determined by an alamarBlue assay. Briefly, primary fibroblasts were seeded overnight in 96-well plates in triplicates and then subjected to serum starvation (0.1% serum) for 24 h. Cells were left untreated or were treated with Glu-plasminogen at the indicated concentrations for 48 h. alamarBlue (resazurin from Sigma) was added to each well at 1.25 μg/ml for 2-4 h, and the fluorometric assay was done with excitation wavelength at 560 nm and emission wavelength at 590 nm with a fluorescence plate reader (FLUOstar Omega, BMG Labtech, Durham, N.C.). For each assay, data were collected from triplicates and analyzed and are represented as the percentage of viable cells relative to the untreated sample.

Caspase-3 protease activity in Glu-plasminogen-treated lung fibroblasts was determined by the fluorometric reaction using acetyl-Asp-Glu-Val-Asp-7-amino-4-trifluoromethylcoumarin (DEVD-AFC; R&D Systems) as substrate according to the manufacturer's instructions. Briefly, following the induction of cell death by Glu-plasminogen for 24 h, cells were collected by centrifugation and lysed in lysis buffer on ice for 10 min. 50 μl of cell lysate (from 2×10⁶ cells) was mixed with 50 μl of 2× reaction buffer and 10 mm dithiothreitol and 5 μl of 1 mm DEVD-AFC in a 96-well plate. After incubation at 37° C. for 90 min, the release of free AFC cleaved by active caspase-3 proteases was determined using the fluorescence microplate reader with excitation at 400 nm and emission at 505 nm. The level of caspase-3 enzymatic activity in the cell lysate is directly proportional to the fluorescent signal of cleaved AFC.

Statistical Analysis. Data are expressed as the mean±S.D. Two factors one-way analysis of variance and Student's t test were used for intergroup comparison. A probability level of 0.05 (p<0.05) was considered significant.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example

### IPF Fibroblasts Constitutively Express Sparc, which Activates B-Catenin, Through Akt-Mediated Inhibition of Glycogen Synthase Kinase 3B (GSK-3B)

To investigate the phenotype of fibroblasts originating from lungs from individuals suffering from idiopathic pulmonary fibrosis (IPF), fibroblasts were isolated from individuals suffering from IPF patients and compared with control fibroblasts, which were isolated from tissue taken at the time of lung cancer resection surgery and remote from the cancer. As reported previously, an increase in total α-SMA was observed in IPF fibroblasts versus control fibroblasts. While examining the expression of known matrix regulatory proteins in fibroblasts isolated from control or IPF lung, which have been shown to regulate the wound-healing response, SPARC expression was found to be increased 8-fold in fibroblasts from IPF lung fibroblasts versus control fibroblasts (J Biol Chem., 2010, 285, 8196-8206). Referring to FIGURE E, enhanced expression of SPARC (secreted protein acidic and rich in cysteine) in IPF fibroblasts stimulated PI3K activity, followed by Akt activation. Akt-mediated phosphorylation of GSK-3β (glycogen synthase kinase-3β) released β-catenin for nuclear translocation and transcriptional activation of PAI-1 (plasminogen activator inhibitor-1). The accumulation of secreted PAI-1 protected IPF fibroblasts from plasminogen-induced apoptosis.

SPARC was more consistently expressed at higher levels in IPF fibroblasts versus control fibroblasts than fibronectin. Type I collagen was consistently expressed at higher levels in IPF fibroblasts, but unlike SPARC, levels varied more with serial passage.

### Example 2

### Dual MTOR Active-Site Inhibitors are more Effective than Rapamycin in Suppressing Constitutive Expression of Mesenchymal Proteins in IPF Fibroblasts and in Response to TGF-β in Normal Lung Fibroblasts

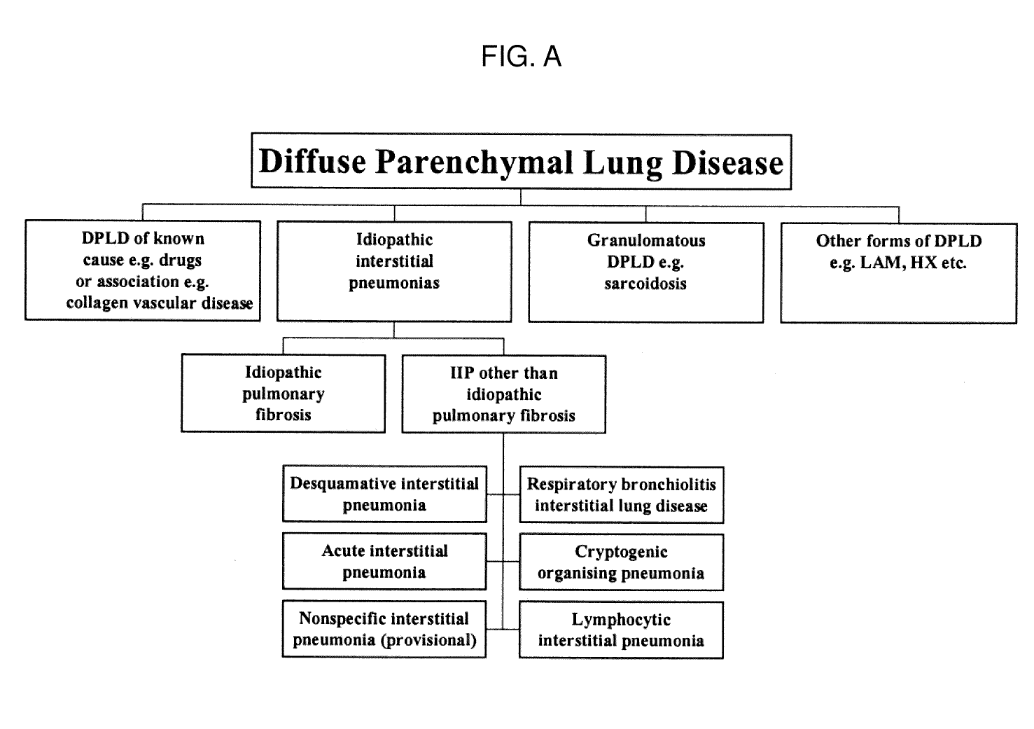

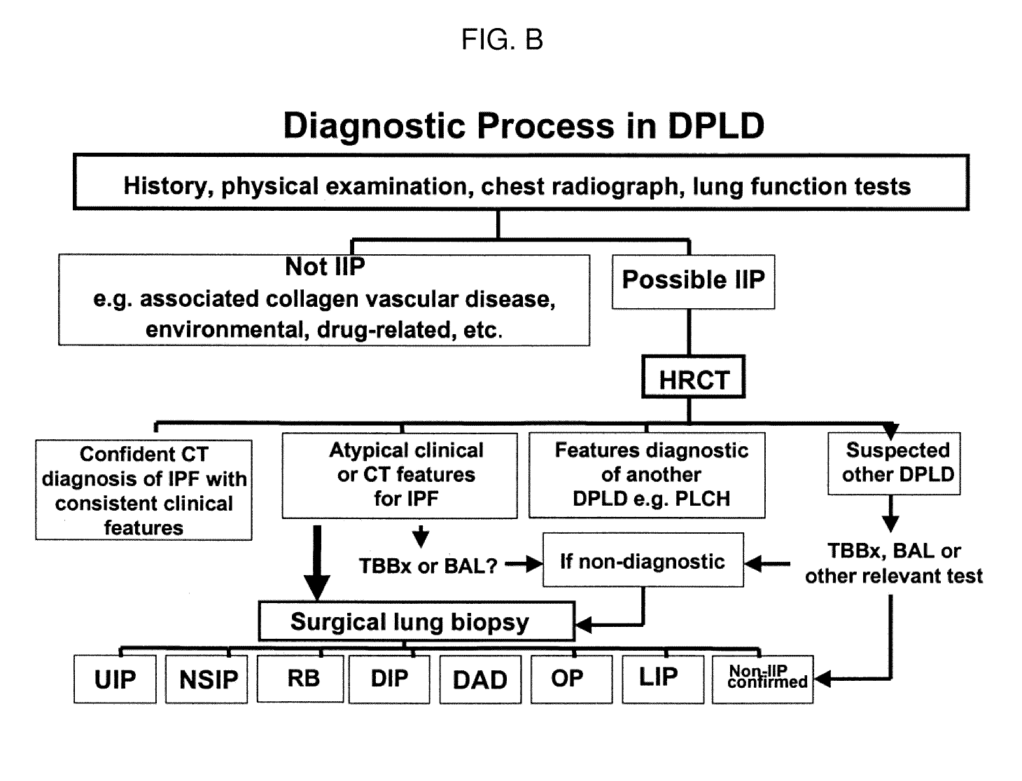

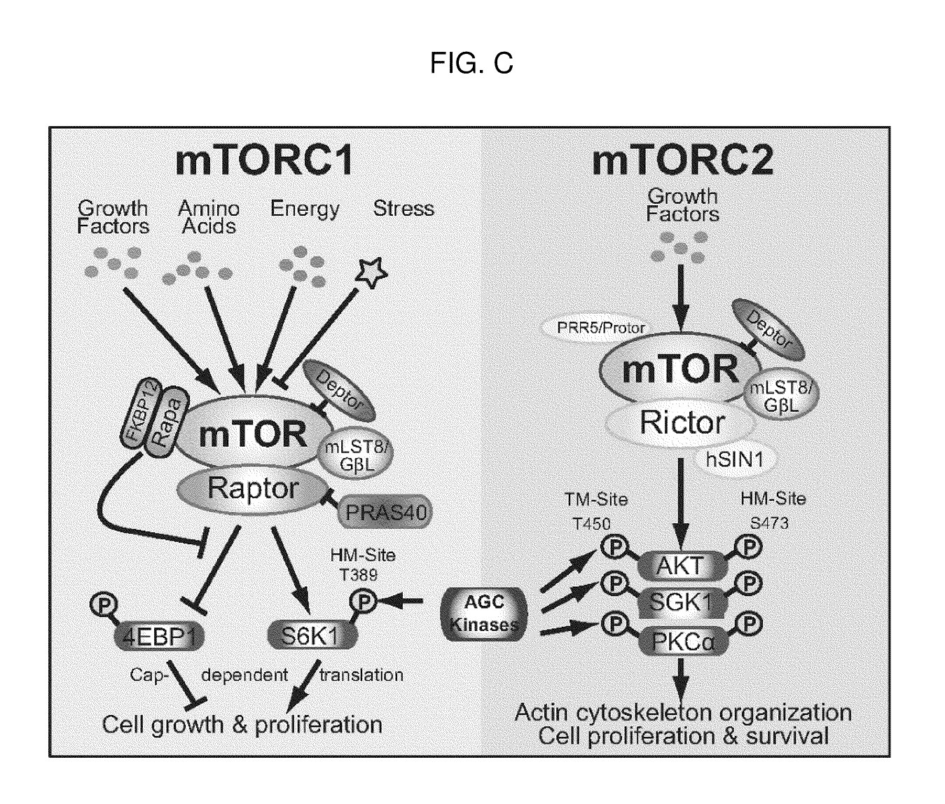

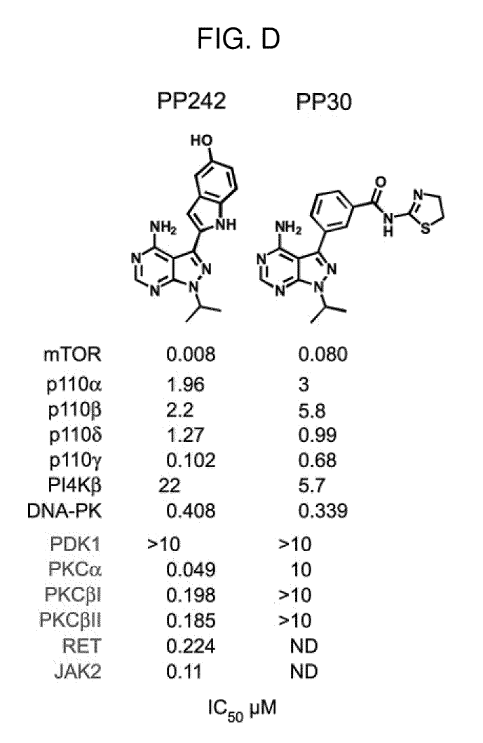

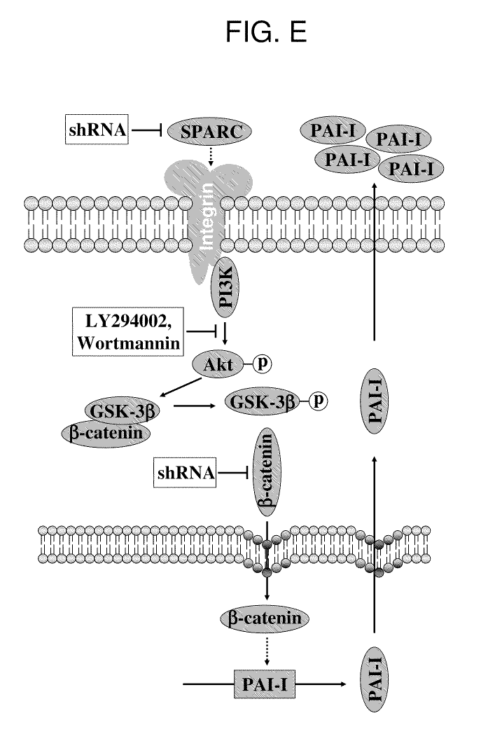

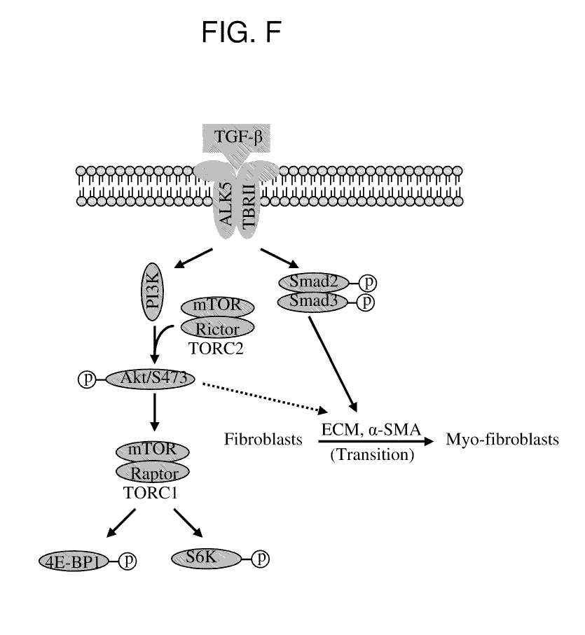

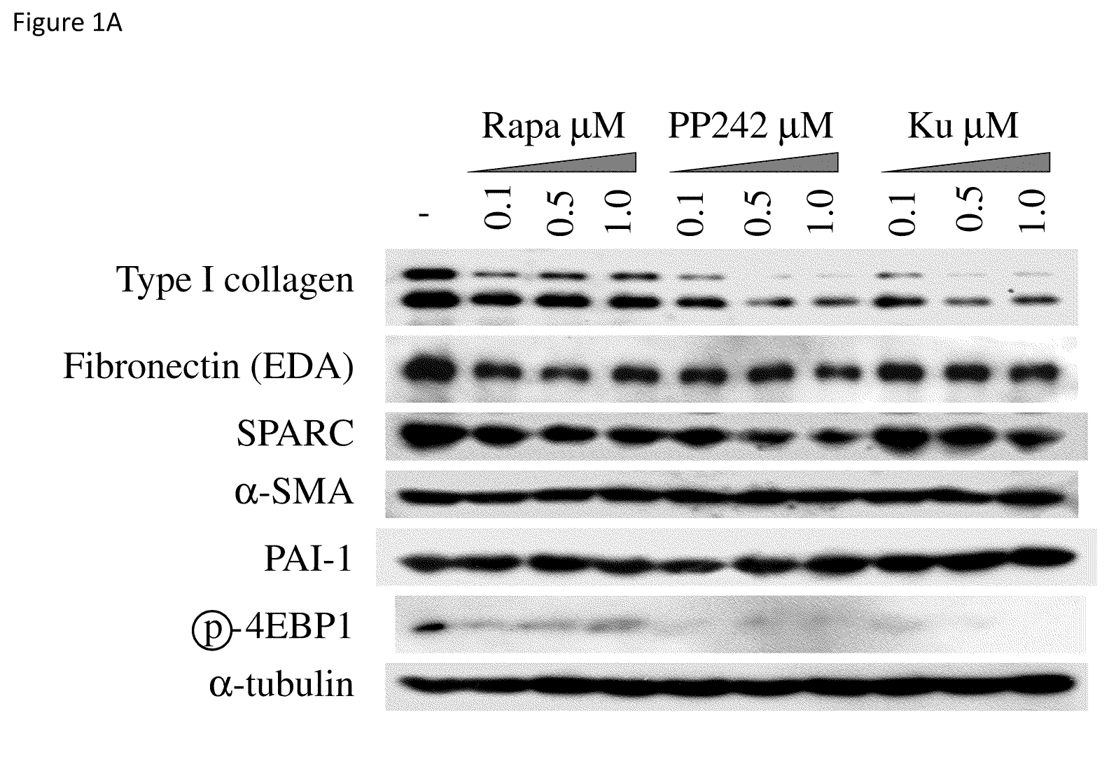

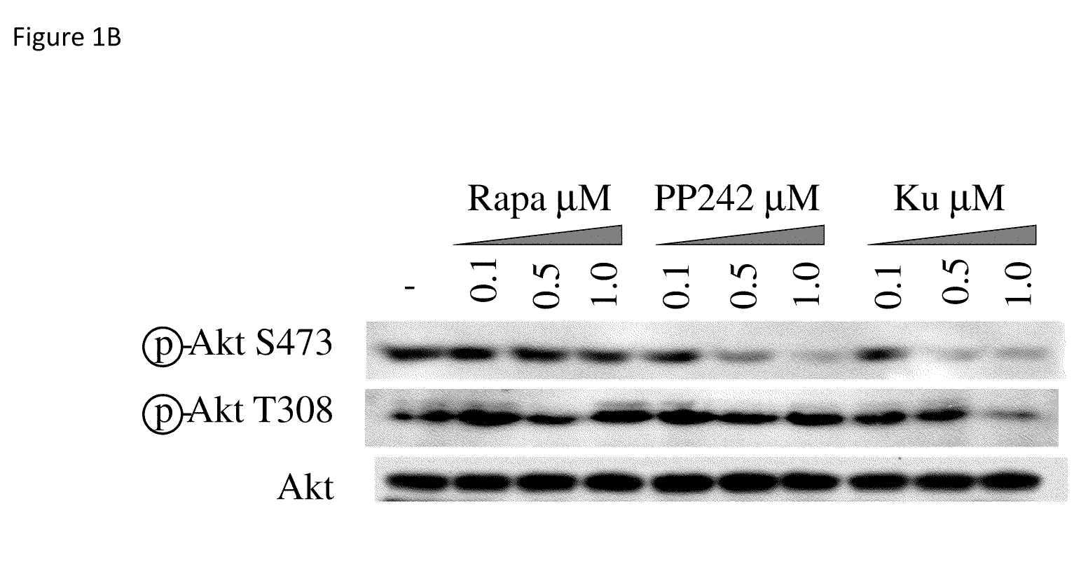

In these studies, we first investigated IPF fibroblasts and found that both PP242 and Ku-0063794, but not rapamycin, suppressed basal Akt (Ser473) phosphorylation in IPF fibroblasts in a dose-dependent manner (FIG. 1A). These doses of rapamycin were 10-100 fold higher than what are used clinically, but mirror the doses that are common for active-site mTOR inhibitors. PP242 and Ku-0063794 were more effective than rapamycin in suppressing basal expression of type I collagen, both inhibiting its expression by over 80% compared to a 50% reduction by rapamycin (FIG. 1A). All inhibitors suppressed FN and SPARC by greater than 50%, but reduced PAI-1 and α-SMA by only 10%-15% of baseline. Cell viability was not compromised by any of these inhibitors at these doses. IPF fibroblasts did not significantly increase stromal protein expression in response to TGF-β, which was likely due to their constitutive high basal expression in IPF fibroblasts (data not shown). All inhibitors blocked the phosphorylation of 4EBP1, which was mTORC1 mediated (FIG. 1A). To establish that Akt (Ser473) phosphorylation was mediated by mTORC2, we showed that PP242 or Ku-0063794, but not rapamycin, inhibited basal Akt (Ser473) phosphorylation in IPF fibroblasts (FIG. 1B). We also looked at their effect on Akt (T308) phosphorylation, which was mediated by PI3K activation of PDK1, and was inhibited by active site mTOR inhibitors (Richard, D. J., J. C. Verheijen, and A. Zask, Recent advances in the development of selective, ATP-competitive inhibitors of mTOR. Curr Opin Drug Discov Devel, 2010. 13(4): p. 428-40.). We found that Ku-0063794 was more effective than rapamycin or PP242 in suppressing basal Akt (T308) phosphorylation in IPF fibroblasts (FIG. 1B).

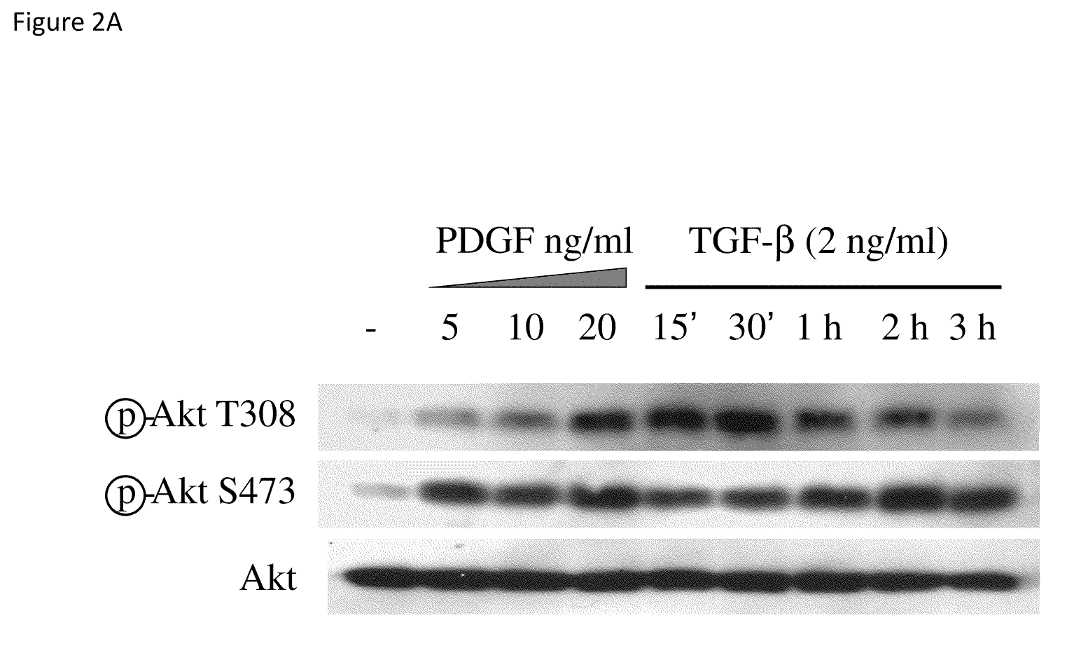

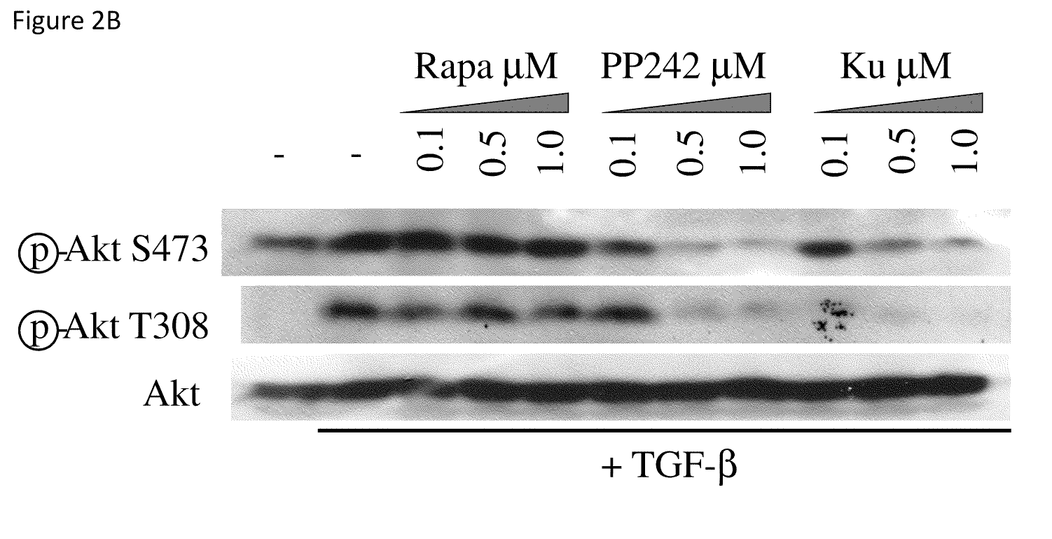

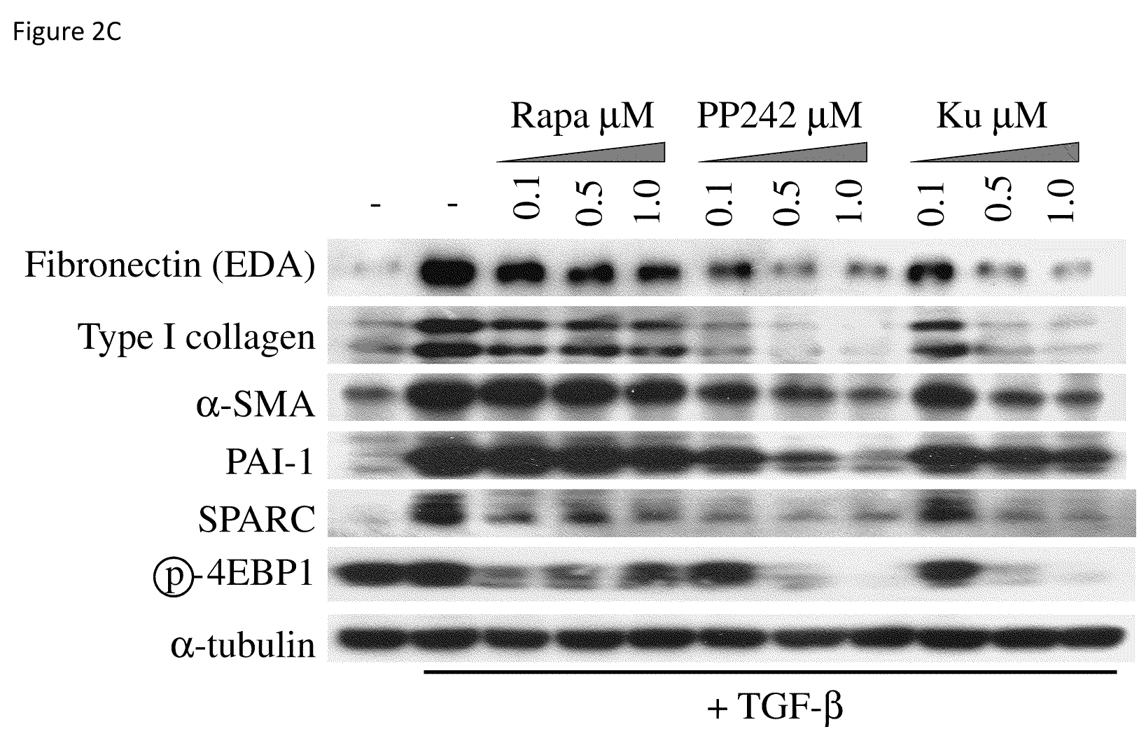

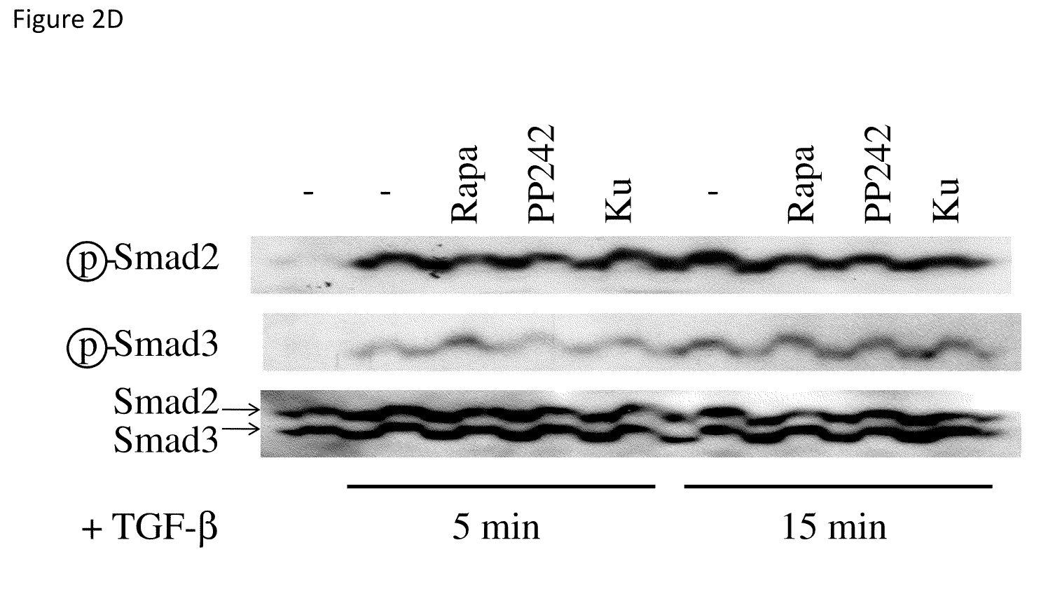

Even though constitutive Akt phosphorylation in IPF fibroblasts does not appear to be mediated by TGF-β alone, there is consensus that TGF-β plays a central role in mediating fibrosis in IPF. In further studies, we examined if inhibition of mTORC1 and/or mTORC2 would limit TGF-β-mediated induction of stromal genes. First, we demonstrated that TGF-β activated Akt in control lung fibroblasts, as we observed activation of both Akt (Ser473) and Akt (T308) at 15′ after TGF-β addition; at 3 h, Akt (Ser473) phosphorylation persisted at similar levels, while Akt (T308) phosphorylation began to decrease (FIG. 2A). PP242 or Ku-0063794, but not rapamycin blocked activation of Akt (Ser473 and T308) (FIG. 2B). We then examined the effect of these inhibitors on TGF-β-mediated induction of stromal genes; as with IPF fibroblasts, we found that PP242 or Ku-0063794 were more effective than rapamycin in blocking expression of type I collagen (FIG. 2C). In fact, PP242 or Ku-0063794 inhibited type I collagen by over 95% in the presence of TGF-β while rapamycin reduced its expression by 45% (FIG. 2C). Also, PP242 or Ku-0063794 blocked induction of FN, SPARC, and α-SMA by 75%-85% while rapamycin inhibited their expression by 30%-50%. Moreover, PP242 inhibited PAI-1 induction by >80% but it was inhibited by 30% in the presence of rapamycin or Ku-0063794 (FIG. 2C). These effects were not mediated by Smad3 or Smad3, as none of the inhibitors blocked TGF-β mediated activation of Smad2 or Smad3 (FIG. 2D).

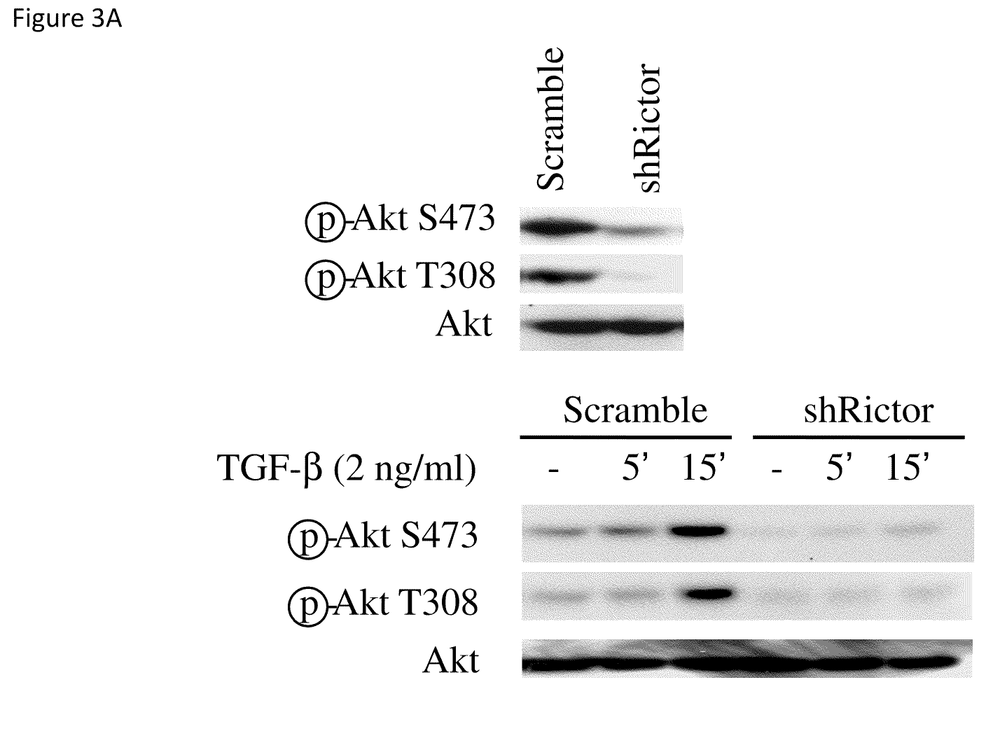

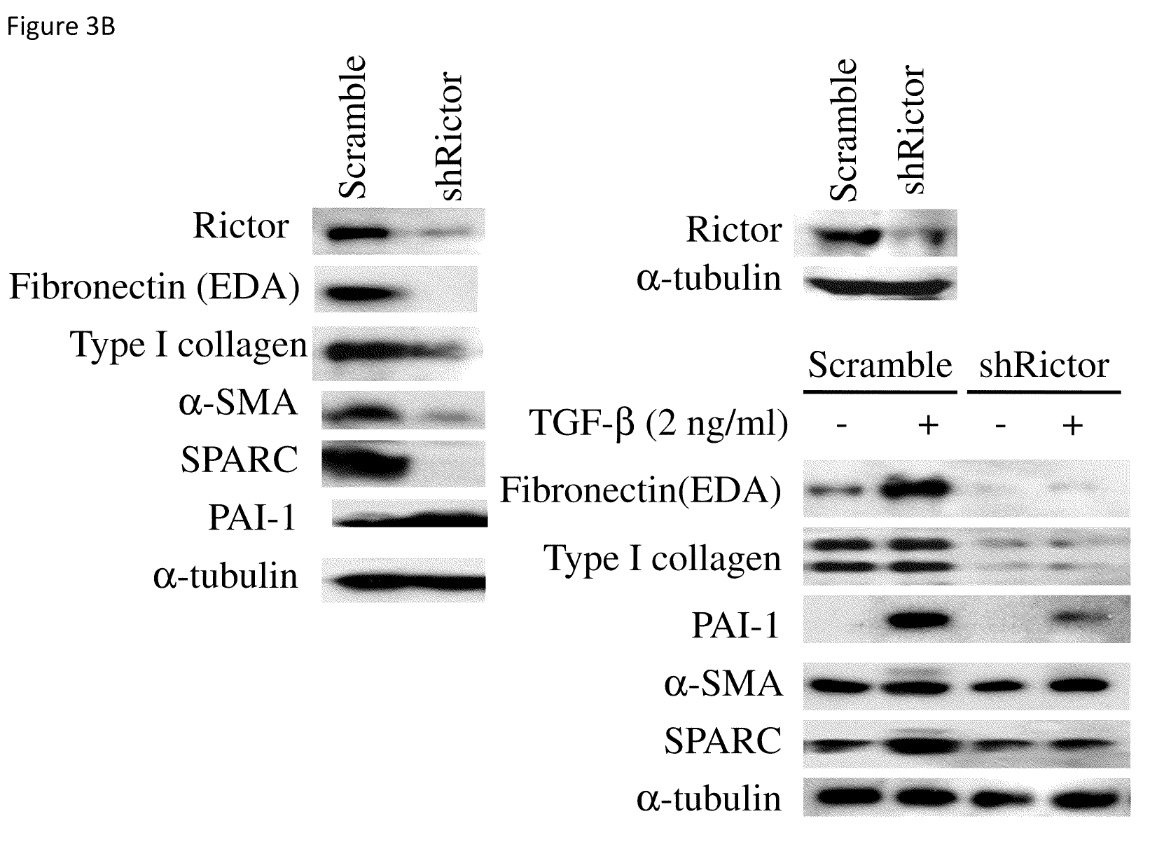

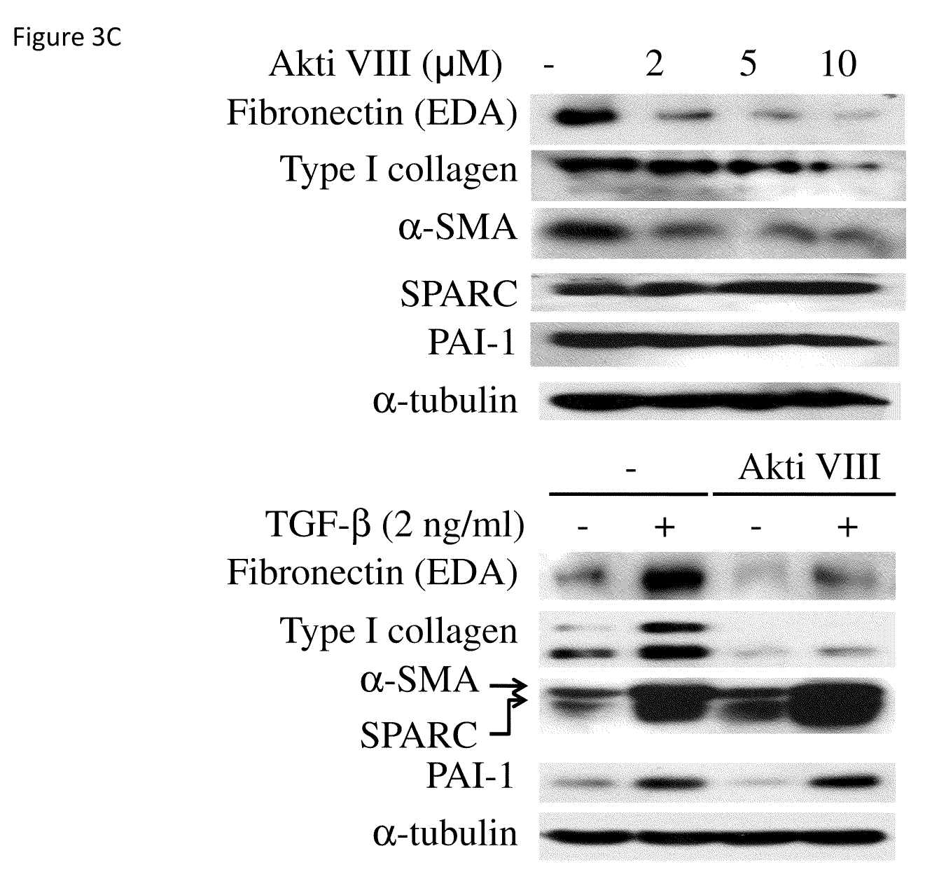

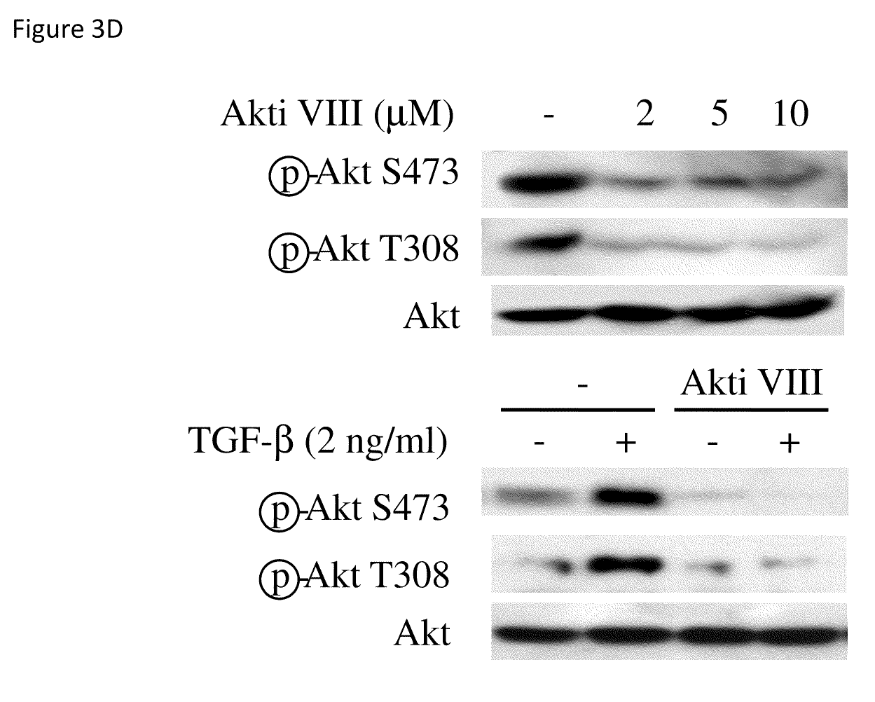

In order to establish that inhibition of mTORC2 mediated, at least in part, suppression of stromal genes constitutively expressed in IPF fibroblasts or induced by TGF-β in control fibroblasts, we examined the effect of downregulation of Rictor by RNA interference, a component of the mTORC2 complex. The downregulation of Rictor suppressed basal Akt phosphorylation (Ser 473 and T308) in IPF fibroblasts and activation by TGF-β in control fibroblasts (FIG. 3A). In IPF fibroblasts, shRictor inhibited FN by 99%, type I collagen by 70%, α-SMA by 95%, and SPARC by 96%; expression of PAI-1, however, was not affected (FIG. 3B). A similar profile was observed in TGF-β-stimulated control fibroblasts except that induction of PAI-1 was inhibited by 80%, but α-SMA was not affected (FIG. 3B). Since Akt is a target of mTORC2, we investigated if inhibition of Akt yielded similar results. We used a specific chemical Akt inhibitor (Akti VIII) and found that it suppressed basal expression of FN, type I collagen, and α-SMA, but not SPARC or PAI-1, in IPF fibroblasts (FIG. 3C); in TGF-β-treated control fibroblasts, Akti VIII inhibited the induction of FN and type I collagen but not α-SMA, SPARC or PAI-1. These results suggest, therefore, that FN and type I collagen are regulated by mTORC2-dependent activation of Akt in IPF fibroblasts but that regulation of SPARC is mediated by Rictor but not Akt; the regulation of PAI-1 or α-SMA is more complex.

In this studies, we show, for the first time, that dual mTOR active-site inhibitors are more effective than rapamycin in suppressing constitutive expression of mesenchymal proteins in IPF fibroblasts and in response to TGF-β in lung normal lung fibroblasts. These data suggest a role for mTORC2 as a mediator of lung fibrosis and suggest that dual mTOR inhibitors, in preference to rapamycin or its analogues, may hold promise for the treatment of fibrotic diseases.

### Example 3

### PP242 Inhibited Bleomycin-Induced Fibrosis when Given Either Simultaneous with Bleomycin or Remote from Bleomycin

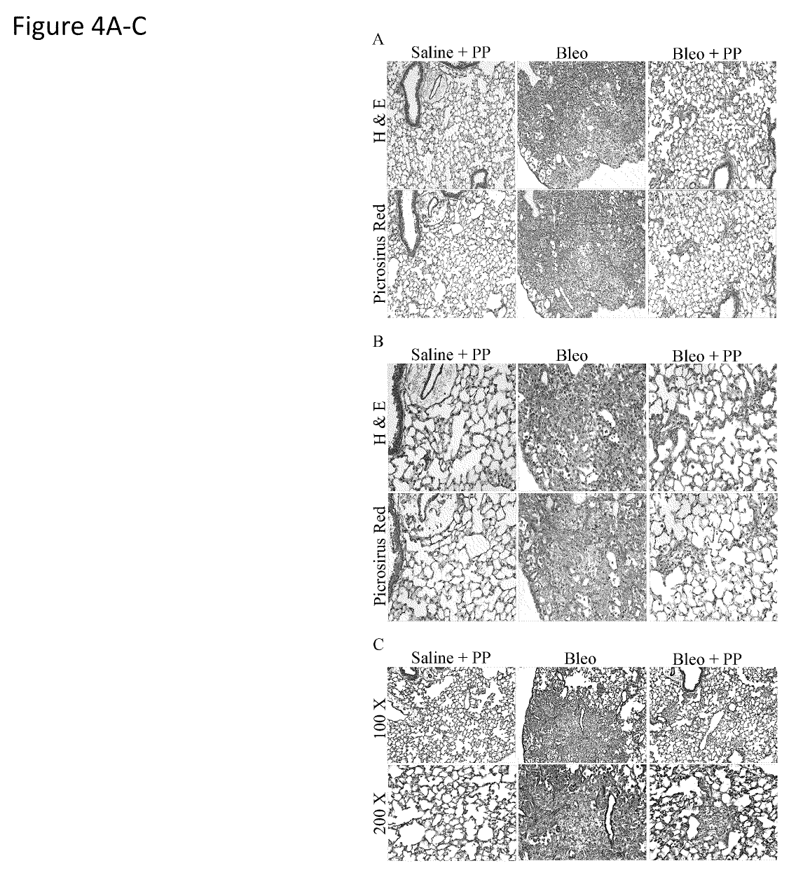

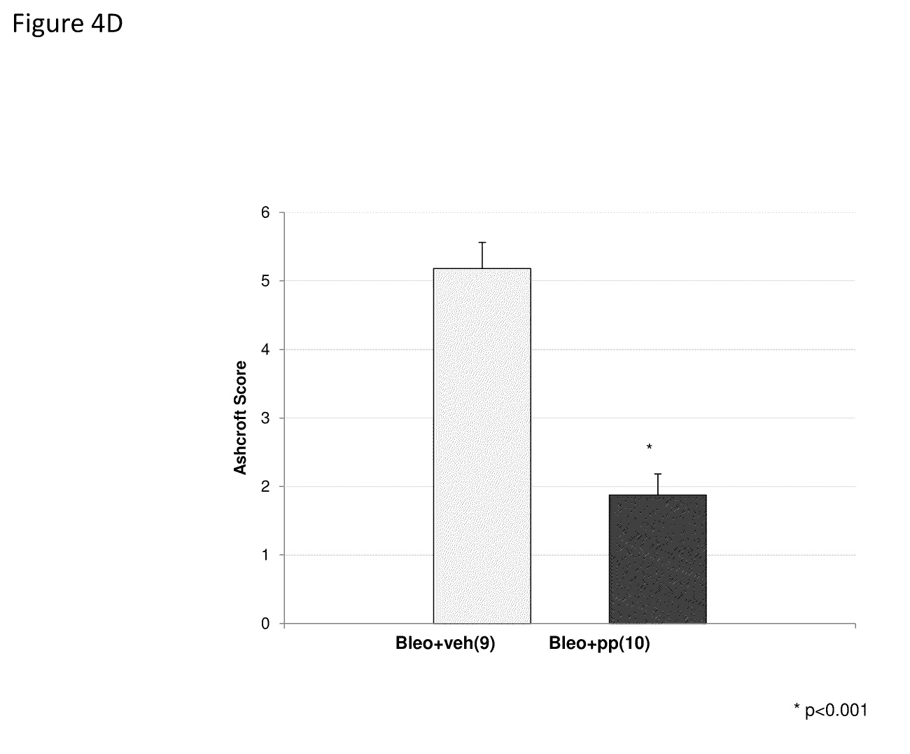

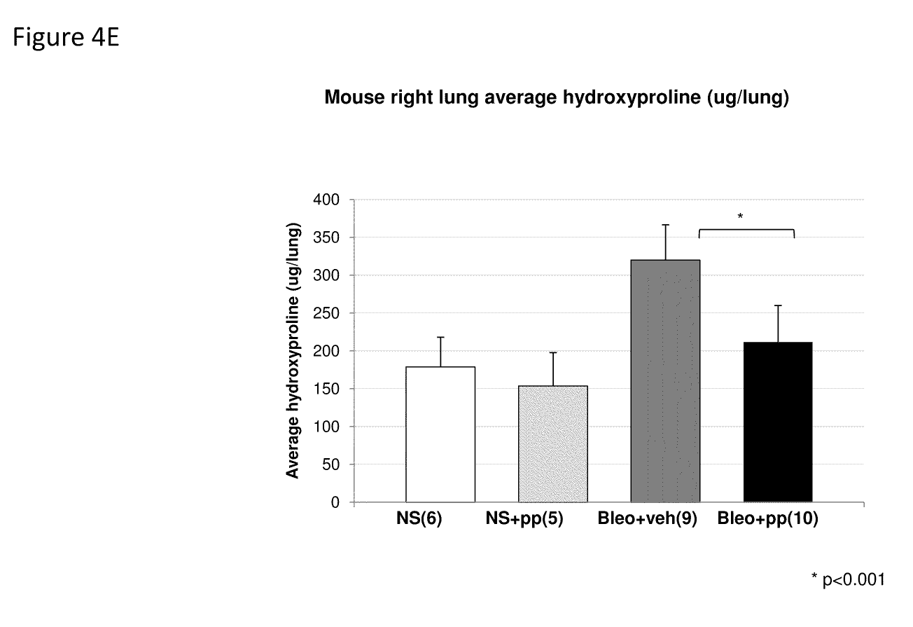

To explore the anti-fibrotic activity of a dual mTOR inhibitor in the murine bleomycin model, we administered PP242 (30 mg/kg) orally, dosages ranging from 30-60 mg/kg, as they have been used in murine cancer models (Shokat). PP242 was given at Day-1 by oral gavage prior to intratracheal instillation of bleomycin (1.5 U/kg), as described previously (Krishna, G., et al., PG490-88, a derivative of triptolide, blocks bleomycin-induced lung fibrosis. Am J Pathol, 2001. 158(3): p. 997-1004) and then daily until sacrifice at Day 14. At the time of harvest, one lung was reserved for histopathologic analysis, i.e., H & E, and Ashcroft score (as assessed by Dr. Gerald Berry, Professor of Pathology, Stanford University); the other lung was used for an analysis of hydroxyproline content. We observed inhibition of bleomycin-induced fibrosis by PP242, as evidenced by a marked reduction in positive staining for picosirius red, a marker of new collagen synthesis (FIG. 4A, 4; also, PP242 inhibited accumulation of α-SMA-positive fibroblasts following bleomycin (FIG. 4C). Also, PP242 plus bleomycin compared to bleomycin alone caused a 70% and 40% reduction in the Ashcroft score and hydroxyproline content respectively (FIG. 4D, 4E). We then examined if genes, which have been shown to mediate fibrosis in the bleomycin model and/or IPF are affected by treatment with PP242; we found that PP242 significantly inhibited bleomycin-mediated induction of SPARC, matrix metalloproteinase 7/matrilysin 1 (MMP-7) and MTS1/S100A4; there was a trend, but not a statistically significant inhibition of PAI-1. We did not observe significant induction of type I collagen, FN, or α-SMA at Day 7 or Day 14 after bleomycin (unpublished results). PP242 was well tolerated; the average weight of animals with PP242 alone was similar to saline control; weight of PP24/bleomycin group was 30-40% more than bleomycin alone (unpublished results).

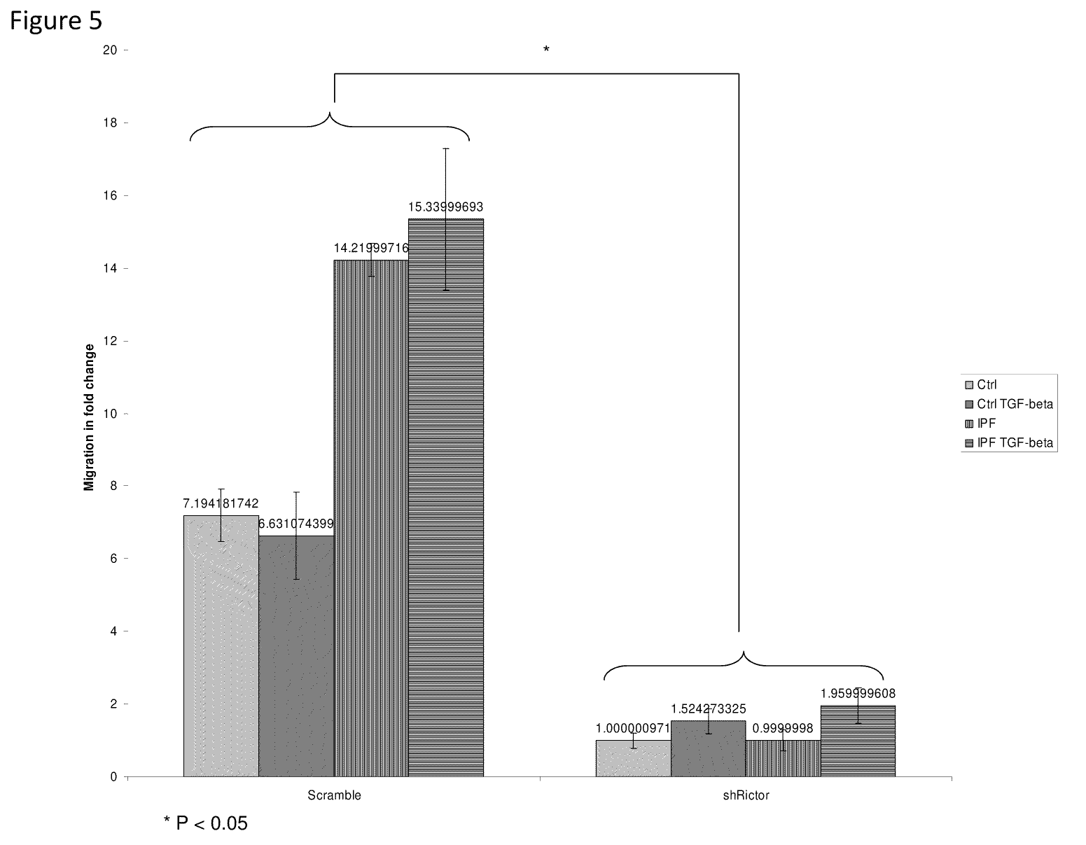

Since the migration of fibroblasts to injured areas is necessary for scar formation in IPF, we investigated whether PP242 or shRictor affected the migration of IPF fibroblasts or control fibroblasts in the absence of presence of TGF-β. We found that PP242 or shRictor significantly reduced the migration of IPF fibroblasts, unstimulated control fibroblasts, or TGF-β-treated fibroblasts; TGF-β did not significantly stimulate migration over baseline, likely because the cells migrated even in serum-depleted conditions (FIG. 5).

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### GENERAL METHODS

Cells Can Be Grown in Culture. The complexity of intact tissues and organs is an inherent disadvantage when trying to extract certain materials. Cells grown in culture provide a more homogeneous population of cells with which to work. Some widely used cell lines are as follows, listing cell line and cell type (and origin): 3T3, fibroblast (mouse); BHK, fibroblast (Syrian hamster); MDCK, epithelial cell (dog); HeLa, epithelial cell (human); PtK1, epithelial cell (rat kangaroo); L6, myoblast (rat); PC12, chromaffin cell (rat); SP2, plasma cell (mouse); COS, kidney (monkey); 293 kidney (human, transformed with adenovirus); CHO, ovary (Chinese hamster); DT40, lymphoma cell for efficient targeted recombination (chick); R1, embryonic stem cell (mouse); E14.1, embryonic stem cell (mouse); H1, H9, embryonic stem cell (human); S2, macrophage-like cell (Drosophila); BY2, undifferentiated meristematic cell (tobacco).

Purifying and Analyzing Proteins

Proteins perform most processes in cells: they catalyze metabolic reactions, use nucleotide hydrolysis to do mechanical work, and serve as the major structural elements of the cell. The great variety of protein structures and functions has stimulated the development of a multitude of techniques to study them.

Proteins Can Be Separated by SDS Polyacrylamide-Gel Electrophoresis

Proteins usually possess a net positive or negative charge, depending on the mixture of charged amino acids they contain. An electric field applied to a solution containing a protein molecule causes the protein to migrate at a rate that depends on its net charge and on its size and shape. The most popular application of this property is SDS polyacrylamide-gel electrophoresis (SDS-PAGE). SDS-PAGE is widely used because it can separate all types of proteins, including those that are normally insoluble in water—such as the many proteins in membranes. And because the method separates polypeptides by size, it provides information about the molecular weight and the subunit composition of proteins. A photograph of a Coomasie-stained gel is handy for memorializing an analysis of each of the successive stages in the purification of a protein.

Specific Proteins Can Be Detected by Blotting with Antibodies

A specific protein can be identified after its fractionation on a polyacrylamide gel by exposing all the proteins present on the gel to a specific antibody that has been coupled to a radioactive isotope, to an easily detectable enzyme, or to a fluorescent dye. For convenience, this procedure is normally carried out after transferring (by “blotting”) all of the separated proteins present in the gel onto a sheet of nitrocellulose paper or nylon membrane. Placing the membrane over the gel and driving the proteins out of the gel with a strong electric field transfers the protein onto the membrane. The membrane is then soaked in a solution of labeled antibody to reveal the protein of interest. This method of detecting proteins is called Western blotting, or immunoblotting.

Mass Spectrometry Provides a Method for Identifying Unknown Proteins

A frequent problem in cell biology and biochemistry is the identification of a protein or collection of proteins that has been obtained by one of the purification procedures for proteins. Because the genome sequences of most common experimental organisms are now known, catalogues of all the proteins produced in those organisms are available. The task of identifying an unknown protein (or collection of unknown proteins) thus reduces to matching some of the amino acid sequences present in the unknown sample with known catalogued genes. This task is now performed almost exclusively by using mass spectrometry in conjunction with computer searches of databases.

Charged particles have very precise dynamics when subjected to electrical and magnetic fields in a vacuum. Mass spectrometry exploits this principle to separate ions according to their mass-to-charge ratio. It is an enormously sensitive technique. It requires very little material and is capable of determining the precise mass of intact proteins and of peptides derived from them by enzymatic or chemical cleavage. Masses can be obtained with great accuracy, often with an error of less than one part in a million. The most commonly used form of the technique is called matrix-assisted laser desorption ionization-time-of-flight spectrometry (MALDI-TOF). In this approach, the proteins in the sample are first broken into short peptides. These peptides are mixed with an organic acid and then dried onto a metal or ceramic slide. A laser then blasts the sample, ejecting the peptides from the slide in the form of an ionized gas, in which each molecule carries one or more positive charges. The ionized peptides are accelerated in an electric field and fly toward a detector. Their mass and charge determines the time it takes them to reach the detector: large peptides move more slowly, and more highly charged molecules move more quickly. By analyzing those ionized peptides that bear a single charge, the precise masses of peptides present in the original sample can be determined. MALDI-TOF can also be used to accurately measure the mass of intact proteins as large as 200,000 daltons. This information is then used to search genomic databases, in which the masses of all proteins and of all their predicted peptide fragments have been tabulated from the genomic sequences of the organism. An unambiguous match to a particular open reading frame can sometimes be made by knowing the mass of only a few peptides derived from a given protein.

MALDI-TOF provides accurate molecular weight measurements for proteins and peptides. Moreover, by employing two mass spectrometers in tandem (an arrangement known as MS/MS), it is possible to directly determine the amino acid sequences of individual peptides in a complex mixture. As described above, the protein sample is first broken down into smaller peptides, which are separated from each other by mass spectrometry. Each peptide is then further fragmented through collisions with high-energy gas atoms. This method of fragmentation preferentially cleaves the peptide bonds, generating a ladder of fragments, each differing by a single amino acid. The second mass spectrometer then separates these fragments and displays their masses. The amino acid sequence of a peptide can then be deduced from these differences in mass.

MS/MS is particularly useful for detecting and precisely mapping post-translational modifications of proteins, such as phosphorylations or acetylations. Because these modifications impart a characteristic mass increase to an amino acid, they are easily detected by mass spectrometry. In combination with rapid purification techniques, mass spectrometry has emerged as a powerful method for detecting post-translational modifications of proteins and the identity of proteins present in mixtures of proteins.

Hydrodynamic Measurements Reveal Size and Shape of a Protein Complex. Most proteins in a cell act as part of larger complexes, and knowledge of the size and shape of these complexes often leads to insights regarding their function. This information can be obtained in several important ways. Sometimes, a complex can be directly visualized using electron microscopy. A complementary approach relies on the hydrodynamic properties of a complex, that is, its behavior as it moves through a liquid medium. Usually, two separate measurements are made. One measure is the velocity of a complex as it moves under the influence of a centrifugal field produced by an ultracentrifuge. The sedimentation constant (or S-value) obtained depends on both the size and the shape of the complex and does not, by itself, convey especially useful information. However, once a second hydrodynamic measurement is performed—by charting the migration of a complex through a gel-filtration chromatography column—both the approximate shape of a complex and its molecular weight can be calculated.

Molecular weight can also be determined more directly by using an analytical ultracentrifuge, a complex device that allows protein absorbance measurements to be made on a sample while it is subjected to centrifugal forces. In this approach, the sample is centrifuged until it reaches equilibrium, where the centrifugal force on a protein complex exactly balances its tendency to diffuse away. Because this balancing point is dependent on a complex's molecular weight but not on its particular shape, the molecular weight can be directly calculated, as needed to determine the stoichiometry of each protein in a protein complex.

Sets of Interacting Proteins Can Be Identified by Biochemical Methods

Because most proteins in the cell function as part of complexes with other proteins, a preliminary way to begin to characterize the biological role of an unknown protein is to identify all of the other proteins to which it specifically binds.

One method for identifying proteins that bind to one another tightly is co-immunoprecipitation. In this case, an antibody recognizes a specific target protein; reagents that bind to the antibody and are coupled to a solid matrix then drag the complex out of solution to the bottom of a test tube. If the original target protein is associated tightly enough with another protein when it is captured by the antibody, the partner precipitates as well. This method is useful for identifying proteins that are part of a complex inside cells, including those that interact only transiently—for example, when extracellular signal molecules stimulate cells. Another method frequently used to identify a protein's binding partners is protein affinity chromatography. To employ this technique to capture interacting proteins, a target protein is attached to polymer beads that are packed into a column. When the proteins in a cell extract are washed through this column, those proteins that interact with the target protein are retained by the affinity matrix. These proteins can then be eluted and their identity determined by mass spectrometry.

In addition to capturing protein complexes on columns or in test tubes, researchers are developing high-density protein arrays to investigate protein interactions. These arrays, which contain thousands of different proteins or antibodies spotted onto glass slides or immobilized in tiny wells, allow one to examine the biochemical activities and binding profiles of a large number of proteins at once. For example, if one incubates a fluorescently labeled protein with arrays containing thousands of immobilized proteins, the spots that remain fluorescent after extensive washing each contain a protein to which the labeled protein specifically binds.

Protein Function Can Be Selectively Modified With Small Molecules. Chemical inhibitors have contributed to the development of cell biology. Small organic molecules are carbon-based compounds that have molecular weights in the range 100-1000 and contain up to 30 or so carbon atoms. In the past, small molecules were usually natural products. The recent development of methods to synthesize hundreds of thousands of small molecules and to carry out large-scale automated screens holds the promise of identifying chemical antagonists and agonists for virtually any biological process. In such approaches, large collections of small chemical compounds are simultaneously tested, either on living cells or in cell-free assays. Once an antagonist or agonist is identified, it can be used as a probe to identify, through affinity chromatography or other means, the protein to which the antagonist or agonist binds and, if antagonism or agonism of protein function is therapeutic, as a drug in and of itself.

Protein Sequence and Structure Provide Clues About Protein Function. Searching a collection of known sequences for similar genes or proteins conventionally involves selecting a database and entering the desired sequence. A sequence alignment program—the most popular are BLAST and FASTA—scans the database for similar sequences by sliding the submitted sequence along the archived sequences until a cluster of residues falls into full or partial alignment. The results of even a complex search—which can be performed on either a nucleotide or an amino acid sequence—are returned within a short time. Such comparisons can predict the functions of individual proteins, families of proteins, or even much of the protein complement of a newly sequenced organism.

Many proteins that adopt the same conformation and have related functions are too distantly related to be identified as clearly similar from a comparison of their amino acid sequences alone. Thus, an ability to reliably predict the three dimensional structure of a protein from its amino acid sequence would improve our ability to infer protein function from the sequence information in genomic databases. In recent years, major progress has been made in predicting the precise structure of a protein. These predictions are based, in part, on our knowledge of tens of thousands of protein structures that have already been determined by x-ray crystallography and NMR spectroscopy and, in part, on computations using our knowledge of the physical forces acting on the atoms. The goal is to predict the structures of proteins that are large or have multiple domains, or predict structures at the very high levels of resolution needed to assist in computer-based drug discovery.

Sequence databases can be searched (or two or more sequences can be aligned) to find similar amino acid or nucleic acid sequences. For example, a BLAST search for proteins similar to the human cell-cycle regulatory protein Cdc2 (Query) locates maize Cdc2 (Sbjct), which is 68% identical (and 82% similar) to human Cdc2 in its amino acid sequence. The alignment begins at residue 57 of the Query protein, suggesting that the human protein has an N-terminal region that is absent from the maize protein. The results of the BLAST search indicate differences in sequence as well as similarities, and when the two amino acid sequences are identical as well as when conservative amino acids are substituted. Here, only one small gap needs to be introduced, at position 194 in the Query sequence, to align the two sequences maximally. The alignment score (Score), which is expressed in two different types of units, takes into account penalties for substitutions and gaps; the higher the alignment score, the better the match. The significance of the alignment is reflected in the Expectation (E) value, which specifies how often a match this good would be expected to occur by chance. The lower the E value, the more significant the match; the very low value in this instance e-111 indicates certain significance. E values much higher than 0.1 are unlikely to reflect true relatedness. For example, an E value of 0.1 means there is a 1 in 10 likelihood that such a match would arise solely by chance.

Protein sequence alignments use standard substitution matrices, for example, the BLOSUM62 matrix, that take into account matches and mismatches of different types (such as a proline to valine, or isoleucine to leucine) based on their different physicochemical and evolutionary properties. Amino acids that are physicochemically similar to one another are determined by their side chains. The common amino acids are grouped according to whether their side chains are acidic, basic, uncharged polar, or nonpolar. Of the 20 amino acids found in proteins, there are equal numbers of polar and non-polar side chains. However, some side chains considered polar are large enough to have some non-polar properties, e.g., Tyr, Thr, Arg, and Lys. Here is the list of amino acids, with their 3 letter abbreviation, 1 letter abbreviation, and grouping by side chain.

| Amino acid | 3 letter name | 1 letter name | Side chain |
|---|---|---|---|
| Aspartic acid | Asp | D | Negative (polar) |
| Glutamic acid | Glu | E | Negative (polar) |
| Arginine | Arg | R | Positive (polar) |
| Lysine | Lys | K | Positive (polar) |
| Histidine | His | H | Positive (polar) |
| Asparagine | Asn | N | Uncharged polar |
| Glutamine | Gln | Q | Uncharged polar |
| Serine | Ser | S | Uncharged polar |
| Threonine | Thr | T | Uncharged polar |
| Tyrosine | Tyr | Y | Uncharged polar |
| Alanine | Ala | A | Nonpolar |
| Glycine | Gly | G | Nonpolar |
| Valine | Val | V | Nonpolar |
| Leucine | Leu | L | Nonpolar |
| Isoleucine | Ile | I | Nonpolar |
| Proline | Pro | P | Nonpolar |
| Phenylalanine | Phe | F | Nonpolar |
| Methionine | Met | M | Nonpolar |
| Tryptophan | Trp | W | Nonpolar |
| Cysteine | Cys | C | Nonpolar |

Generally speaking, one requires a 30% identity in sequence to consider that two polypeptides match. While finding similar sequences and structures for a new protein will provide many clues about its function, it may be necessary to test these insights through direct experimentation. However, the clues generated from sequence comparisons traditionally point the investigator in the correct experimental direction. The use of sequence alignments has therefore become one of the choicest strategies in modern cell biology.

Analyzing and Manipulating DNA. Central to the technology are the following key techniques: 1. Cleavage of DNA at specific sites by restriction nucleases, which greatly facilitates the isolation and manipulation of individual genes. 2. DNA ligation, which makes it possible to design and construct DNA molecules that are not found in nature. 3. DNA cloning through the use of either cloning vectors or the polymerase chain reaction, in which a portion of DNA is repeatedly copied to generate many billions of identical molecules. 4. Nucleic acid hybridization, which makes it possible to find a specific sequence of DNA or RNA with great accuracy and sensitivity on the basis of its ability to selectively bind a complementary nucleic acid sequence. 5. Determination of the sequence of nucleotides of any DNA (even entire genomes), making it possible to identify genes and to deduce the amino acid sequence of the proteins they encode. 6. Simultaneous monitoring of the level of mRNA produced by genes in a cell using nucleic acid microarrays, in which tens of thousands of hybridization reactions take place simultaneously.

Nucleic Acid Hybridization Detects Specific Nucleotide Sequences. When an aqueous solution of DNA is heated at 100° C. or exposed to a very high pH (pH≧13), the complementary base pairs that normally hold the two strands of the double helix together are disrupted and the double helix rapidly dissociates into two single strands. This process, called DNA denaturation, was for many years thought to be irreversible. It was discovered, however, that complementary single strands of DNA readily re-form double helices by a process called hybridization (also called DNA renaturation) if they are kept for a prolonged period at 65° C. Similar hybridization reactions can occur between any two single-stranded nucleic acid chains (DNA/DNA, RNA/RNA, or RNA/DNA), provided that they have complementary nucleotide sequences. These specific hybridization reactions are widely used to detect and characterize specific nucleotide sequences in both RNA and DNA molecules.

Single-stranded DNA molecules used to detect complementary sequences are known as probes; these molecules, which carry radioactive or chemical markers to facilitate their detection, can range from fifteen to thousands of nucleotides long. Hybridization reactions using DNA probes are so sensitive and selective that they can detect complementary sequences present at a concentration as low as one molecule per cell. It is thus possible to determine how many copies of any DNA sequence are present in a particular DNA sample. The same technique can be used to search for similar but nonidentical genes. To find a gene of interest in an organism whose genome has not yet been sequenced, for example, a portion of a known gene can be used as a probe.

Stringent versus nonstringent hybridization conditions tell sequences apart. To use a DNA probe to find an almost identical match, high stringent hybridization conditions are used; the reaction temperature is kept just a few degrees below that at which a perfect DNA helix denatures in the solvent used (its melting temperature), so that all imperfect helices formed are unstable. Lowering the salt concentration lowers the melting point, as does the addition of formamide. As an example, hybridization is in 50% formamide at 42° C. When a DNA probe is being used to find DNAs with similar, as well as identical, sequences, low stringent conditions are used; hybridization is performed at a lower temperature, which allows even imperfectly paired double helices to form. Continuing with this example, hybridization is in 50% formamide at 35° C. The lower temperature hybridization conditions are used to search for genes that are nonidentical but similar.

Alternatively, DNA probes can be used in hybridization reactions with RNA rather than DNA to find out whether a cell is expressing a given gene. In this case a DNA probe that contains part of the gene's sequence is hybridized with RNA purified from the cell in question to see whether the RNA includes nucleotide sequences matching the probe DNA and, if so, in what quantities. In somewhat more elaborate procedures, the DNA probe is treated with specific nucleases after the hybridization is complete, to determine the exact regions of the DNA probe that have paired with the RNA molecules. One can thereby determine the start and stop sites for RNA transcription, as well as the precise boundaries of the intron and exon sequences in a gene.

Today, the positions of intron/exon boundaries are usually determined by sequencing the complementary DNA (cDNA) sequences that represent the mRNAs expressed in a cell and comparing them with the nucleotide sequence of the genome. We describe later how cDNAs are prepared from mRNAs.

The hybridization of DNA probes to RNAs allows one to determine whether or not a particular gene is being transcribed; moreover, when the expression of a gene changes, one can determine whether the change is due to transcriptional or post-transcriptional controls. These tests of gene expression were initially performed with one DNA probe at a time. DNA microarrays now allow the simultaneous monitoring of hundreds or thousands of genes at a time. Hybridization methods are still in wide use in cell biology today.

Blotting Facilitates Hybridization with Separated Nucleic Acid Molecules. Specific RNA or DNA molecules are detected by gel-transfer hybridization in a method called Southern blotting (named after its inventor) or Northern blotting (named with reference to Southern blotting). To start, one collects tissue from a source and disrupts the cells in a strong detergent to inactivate nucleases that might otherwise degrade the nucleic acids. Next, one separates the RNA and DNA from all of the other cell components: the proteins present are completely denatured and removed by repeated extractions with phenol—a potent organic solvent that is partly miscible with water; the nucleic acids, which remain in the aqueous phase, are then precipitated with alcohol to separate them from the small molecules of the cell. Then one separates the DNA from the RNA by their different solubilities in alcohols and degrades any contaminating nucleic acid of the unwanted type by treatment with a highly specific enzyme—either an RNase or a DNase. The mRNAs are typically separated from bulk RNA by retention on a chromatography column that specifically binds the poly-A tails of mRNAs.

DNA probes detected by chemical or fluorescence methods or radioactivity are widely used. First, a mixture of either single-stranded RNA molecules (Northern blotting) or the double-stranded DNA fragments created by restriction nuclease treatment (Southern blotting) is separated according to length by electrophoresis. Next, a sheet of nitrocellulose or nylon paper is laid over the gel, and the separated RNA or DNA fragments are transferred to the sheet by blotting. Then, the nitrocellulose sheet is carefully peeled off the gel. Next, the sheet containing the bound nucleic acids is placed in a sealed plastic bag together with a buffered salt solution containing a radioactively labeled DNA probe. The sheet is exposed to a labeled DNA probe for a prolonged period under conditions favoring hybridization. Last, the sheet is removed from the bag and washed thoroughly, so that only probe molecules that have hybridized to the RNA or DNA immobilized on the paper remain attached. After autoradiography, the DNA that has hybridized to the labeled probe shows up as bands on the autoradiograph. For Southern blotting, the strands of the double-stranded DNA molecules on the paper must be separated before the hybridization process; this is done by exposing the DNA to alkaline denaturing conditions after the gel has been run.

Genes Can Be Cloned Using DNA Libraries

Genes can be cloned using DNA libraries. Almost any DNA fragment can be cloned. In molecular biology, the term DNA cloning is used in two senses. In one sense, it literally refers to the act of making many identical copies of a DNA molecule—the amplification of a particular DNA sequence. However, the term also describes the isolation of a particular stretch of DNA (often a particular gene) from the rest of a cell's DNA, because this isolation is greatly facilitated by making many identical copies of the DNA of interest. In both cases, cloning refers to the act of making many genetically identical copies.

DNA cloning in its most general sense can be accomplished in several ways. The simplest involves inserting a particular fragment of DNA into the purified DNA genome of a self-replicating genetic element—generally a virus or a plasmid. A DNA fragment containing a human gene, for example, can be joined in a test tube to the chromosome of a bacterial virus, and the new recombinant DNA molecule can then be introduced into a bacterial cell, where the inserted DNA fragment will be replicated along with the DNA of the virus. Starting with only one such recombinant DNA molecule that infects a single cell, the normal replication mechanisms of the virus can produce more than 10 to the power of 12 identical virus DNA molecules in a single day, thereby amplifying the amount of the inserted human DNA fragment by the same factor. A virus or plasmid used in this way is known as a cloning vector, and the DNA propagated by insertion into it is said to have been cloned.

To isolate a specific gene, one begins by constructing a DNA library—a comprehensive collection of cloned DNA fragments from a cell, tissue, or organism. This library includes (one hopes) at least one fragment that contains the gene of interest. Libraries can be constructed with either a virus or a plasmid vector and are generally housed in a population of bacterial cells. The principles underlying the methods used for cloning genes are the same for either type of cloning vector, although the details may differ. Today, most cloning is performed with plasmid vectors.

The plasmid vectors most widely used for gene cloning are small circular molecules of double-stranded DNA derived from larger plasmids that occur naturally in bacterial cells. They generally account for only a minor fraction of the total host bacterial cell DNA, but they can easily be separated owing to their small size from chromosomal DNA molecules, which are large and precipitate as a pellet upon centrifugation. For use as cloning vectors, the purified plasmid DNA circles are first cut with a restriction nuclease to create linear DNA molecules. The genomic DNA to be used in constructing the library is cut with the same restriction nuclease, and the resulting restriction fragments (including those containing the gene to be cloned) are then added to the cut plasmids and annealed via their cohesive ends to form recombinant DNA circles. These recombinant molecules containing foreign DNA inserts are then covalently sealed with the enzyme DNA ligase.

In the next step in preparing the library, the recombinant DNA circles are introduced into bacterial cells that have been made transiently permeable to DNA. These bacterial cells are now said to be transfected with the plasmids. As the cells grow and divide, doubling in number every 30 minutes, the recombinant plasmids also replicate to produce an enormous number of copies of DNA circles containing the foreign DNA. Many bacterial plasmids carry genes for antibiotic resistance, a property that can be exploited to select those cells that have been successfully transfected; if the bacteria are grown in the presence of the antibiotic, only cells containing plasmids will survive. Each original bacterial cell that was initially transfected contains, in general, a different foreign DNA insert; this insert is inherited by all of the progeny cells of that bacterium, which together form a small colony in a culture dish.

For many years, plasmids were used to clone fragments of DNA of 1000 to 30,000 nucleotide pairs. Larger DNA fragments are more difficult to handle and were harder to clone. Today, new plasmid vectors based on the naturally occurring F plasmid of E. coli are used to clone DNA fragments of 300,000 to 1 million nucleotide pairs. Unlike smaller bacterial plasmids, the F plasmid—and its derivative, the bacterial artificial chromosome (BAC)—is present in only one or two copies per E. coli cell. The fact that BACs are kept in such low numbers in bacterial cells may contribute to their ability to maintain large cloned DNA sequences stably: with only a few BACs present, it is less likely that the cloned DNA fragments will become scrambled by recombination with sequences carried on other copies of the plasmid. Because of their stability, ability to accept large DNA inserts, and ease of handling, BACs are now the preferred vector for building DNA libraries of complex organisms—including those representing the human genome.

Two Types of DNA Libraries Serve Different Purposes

Cleaving the entire genome of a cell with a specific restriction nuclease and cloning each fragment as just described produces a very large number of DNA fragments—on the order of a million for a mammalian genome. The fragments are distributed among millions of different colonies of transfected bacterial cells. Each of the colonies is composed of a clone of cells derived from a single ancestor cell, and therefore harbors many copies of a particular stretch of the fragmented genome. Such a plasmid is said to contain a genomic DNA clone, and the entire collection of plasmids is called a genomic DNA library. But because the genomic DNA is cut into fragments at random, only some fragments contain genes. Many of the genomic DNA clones obtained from the DNA of a higher eukaryotic cell contain only noncoding DNA, which makes up most of the DNA in such genomes.

An alternative strategy is to begin the cloning process by selecting only those DNA sequences that are transcribed into mRNA and thus are presumed to correspond to protein-encoding genes. This is done by extracting the mRNA from cells and then making a DNA copy of each mRNA molecule present—a so-called complementary DNA, or cDNA. The copying reaction is catalyzed by the reverse transcriptase enzyme of retroviruses, which synthesizes a complementary DNA chain on an RNA template. The single-stranded cDNA molecules synthesized by the reverse transcriptase are converted into double-stranded cDNA molecules by DNA polymerase, and these molecules are inserted into a plasmid or virus vector and cloned. Each clone obtained in this way is called a cDNA clone, and the entire collection of clones derived from one mRNA preparation constitutes a cDNA library.

cDNA Clones Contain Uninterrupted Coding Sequences

There are some important differences between genomic DNA clones and cDNA clones. Genomic clones represent a random sample of all the DNA sequences in an organism and, with very rare exceptions, are the same regardless of the cell type used to prepare them. By contrast, cDNA clones contain only those regions of the genome that have been transcribed into mRNA. Because the cells of different tissue types produce distinct sets of mRNA molecules, a distinct cDNA library is obtained for each type of cell used to prepare the library.

The most important advantage of cDNA clones is that they contain the uninterrupted coding sequence of a gene. Eukaryotic genes usually consist of short coding sequences of DNA (exons) separated by much longer noncoding sequences (introns); the production of mRNA entails the removal of the noncoding sequences from the initial RNA transcript and the splicing together of the coding sequences. Bacterial cells will not make these modifications to the RNA produced from a gene of a higher eukaryotic cell. Thus, when the aim of the cloning is either to deduce the amino acid sequence of the protein from the DNA sequence or to produce the protein in bulk by expressing the cloned gene in a bacterial cell, it is much preferable to start with cDNA. cDNA libraries have the additional advantage of representing alternatively spliced mRNAs produced from a given cell or tissue.

Genomic and cDNA libraries are widely shared among investigators and, today, many such libraries are also available from commercial sources.

Genes Can Be Selectively Amplified by PCR

Now that so many genome sequences are available, genes can be cloned directly without the need to first construct DNA libraries. A technique called polymerase chain reaction (PCR) makes this rapid cloning possible. Starting with an entire genome, PCR allows the DNA from a selected region to be amplified several billionfold, effectively “purifying” this DNA away from the remainder of the genome.

To begin, a pair of DNA oligonucleotides, chosen to flank the desired nucleotide sequence of the gene, are synthesized by chemical methods. These oligonucleotides are then used to prime DNA synthesis on single strands generated by heating the DNA from the entire genome. The newly synthesized DNA is produced in a reaction catalyzed in vitro by a purified DNA polymerase, and the primers remain at the 5′ ends of the final DNA fragments that are made.

Nothing special is produced in the first cycle of DNA synthesis; the power of the PCR method is revealed only after repeated rounds of DNA synthesis. Every cycle doubles the amount of DNA synthesized in the previous cycle. Because each cycle requires a brief heat treatment to separate the two strands of the template DNA double helix, the technique requires the use of a special DNA polymerase, isolated from a thermophilic bacterium, that is stable at much higher temperatures than normal so that it is not denatured by the repeated heat treatments. With each round of DNA synthesis, the newly generated fragments serve as templates in their turn, and within a few cycles the predominant product is a single species of DNA fragment whose length corresponds to the distance between the two original primers.

In practice, effective DNA amplification requires 20-30 reaction cycles, with the products of each cycle serving as the DNA templates for the next—hence the term polymerase “chain reaction.” A single cycle requires only about 5 minutes, and the entire procedure can be easily automated. PCR thereby makes possible the “cell-free molecular cloning” of a DNA fragment in a few hours, compared with the several days for standard cloning procedures. This technique is now used routinely to clone DNA from genes of interest directly—starting either from genomic DNA or from mRNA isolated from cells.

The PCR method is extremely sensitive; it can detect a single DNA molecule in a sample. Trace amounts of RNA can be analyzed in the same way by first transcribing them into DNA with reverse transcriptase. The PCR cloning technique has largely replaced Southern blotting for the diagnosis of genetic diseases and for the detection of low levels of viral infection.

Expression of Genes Can Be Measured Using Quantitative RT-PCR

It is often desirable to quantitate gene expression by directly measuring mRNA levels in cells. Although Northern blots can be adapted to this purpose, a more accurate method is based on the principles of PCR. This method, called quantitative RT-PCR (reverse transcription-polymerase chain reaction), begins with the total population of mRNA molecules purified from a tissue or a cell culture. It is important that no DNA be present in the preparation; it must be purified away or enzymatically degraded. Two DNA primers that specifically match the gene of interest are added, along with reverse transcriptase, DNA polymerase, and the four deoxynucleoside triphosphates needed for DNA synthesis. The first round of synthesis is the reverse transcription of the mRNA into DNA using one of the primers. Next, a series of heating and cooling cycles allows the amplification of that DNA strand by conventional PCR. The quantitative part of this method relies on a direct relationship between the rate at which the PCR product is generated and the original concentration of the mRNA species of interest. By adding chemical dyes to the PCR reaction that fluoresce only when bound to double-stranded DNA, a simple fluorescence measurement can be used to track the progress of the reaction and thereby accurately deduce the starting concentration of the mRNA that is amplified. Although it seems complicated, this quantitative RT-PCR technique (sometimes called real time PCR) is straightforward to perform in the laboratory; it has displaced Northern blotting as the method of choice for quantifying mRNA levels from any given gene.

Cells Can Be Used As Factories to Produce Specific Proteins

The vast majority of the thousands of different proteins in a cell, including many with crucially important functions, are present in very small amounts. In the past, for most of them, it has been extremely difficult, if not impossible, to obtain more than a few micrograms of pure material. One of the most important contributions of DNA cloning and genetic engineering to cell biology is that they have made it possible to produce almost any of the cell's proteins in a nearly unlimited amount.

Large amounts of the desired protein are produced in living cells by using expression vectors. These are generally plasmids that have been designed to produce a large amount of a stable mRNA that can be efficiently translated into protein in the transfected bacterial, yeast, insect, or mammalian cell. A plasmid vector is engineered to contain a highly active promoter, which causes unusually large amounts of mRNA to be produced from an adjacent protein-coding gene inserted into the plasmid vector. Depending on the characteristics of the cloning vector, the plasmid is introduced into bacterial, yeast, insect, or mammalian cells, where the inserted gene is efficiently transcribed and translated into protein.

Because the desired protein made from an expression vector is produced inside a cell, it must be purified away from the host-cell proteins by chromatography after cell lysis; but because it is a plentiful species in the cell lysate (often 1-10% of the total cell protein), the purification is usually easy to accomplish in only a few steps. In order to purify a protein, it first must be extracted from inside the cell, unless it is secreted into the medium. The cells are typically homogenized to produce a homogenate or slurry. The homogenate is typically fractionated into different components by centrifugation. After centrifugation, proteins are often separated by chromatography. Secreted, soluble proteins are isolated from the supernatants of infected cells after pelleting the cells by centrifugation and do not require cell lysis. A variety of expression vectors are available, each engineered to function in the type of cell in which the protein is to be made. In this way, cells can be induced to make vast quantities of proteins useful for medical purposes or to be studied for structure and function.

Genes Can Be Engineered by Site-Directed Mutagenesis

A technique called site-directed mutagenesis changes selected amino acids in a protein. To begin, a recombinant plasmid containing a gene insert is separated into its two DNA strands. A synthetic oligonucleotide primer corresponding to part of the gene sequence but containing a single altered nucleotide at a predetermined point is added to the single-stranded DNA under conditions that permit imperfect DNA hybridization. The primer hybridizes to the DNA, forming a single mismatched nucleotide pair. The recombinant plasmid is made double-stranded by in vitro DNA synthesis (starting from the primer) followed by sealing by DNA ligase. The double-stranded DNA is introduced into a cell, where it is replicated. Replication using one strand of the template produces a normal DNA molecule, but replication using the other strand (the one that contains the primer) produces a DNA molecule carrying the desired mutation. Only half of the progeny cells will end up with a plasmid that contains the desired mutant gene. However, a progeny cell that contains the mutated gene can be identified, separated from other cells, and cultured to produce a pure population of cells, all of which carry the mutated gene. With an oligonucleotide of the appropriate sequence, more than one amino acid substitution can be made at a time, or one or more amino acids can be inserted or deleted. It is also possible to create a site-directed mutation by using the appropriate oligonucleotides and PCR (instead of plasmid replication) to amplify the mutated gene.

Proteins and Nucleic Acids Can Be Synthesized By Chemical Reactions

Chemical reactions have been devised to synthesize directly specific sequences of amino acids or nucleic acids. These methodologies provide direct sources of biological molecules and do not rely on any cells or enzymes. Chemical synthesis is the method of choice for obtaining nucleic acids in the range of 100 nucleotides or fewer, which, under the basic concept of de novo gene synthesis, may be assembled into larger constructs using some form of polymerase chain assembly or ligase chain reaction approach. Chemical synthesis is also routinely used to produce specific peptides that, when chemically coupled to other proteins, are used to generate antibodies against the peptide.

Pharmaceutical Manufacturing and Administration

Pharmaceutical Solids. The discovery and development of new chemical entities (NCEs) into stable, bioavailable, marketable pharmaceutical compositions is a long process. Due to the tremendous cost of developing a NCE, and industry's need to enhance productivity, it is desirable to create NCEs that have suitable physical-chemical properties, rather than compensate for deficiencies solely by the formulation process. Hence, property-based design can enhance the likelihood a NCE will have the desired physical-chemical properties that will facilitate its ability to be developed into a stable, bioavailable dosage form. Even so, well-designed preformulation studies are necessary to fully characterize molecules during the discovery and development process so that NCEs have the appropriate properties, and there is an understanding of the deficiencies that must be overcome by the formulation process.

Once a NCE is selected for development, choosing the molecular form that will be the active pharmaceutical ingredient (API) is the next milestone. Salt selection is the first API decision, in which absorption needs to be balanced with consistency of the API solid state. Excipients are the backbone of a formulation; they may be needed to stabilize the API.

A wide variety of different solid states are possible. Polymorphs exist when the drug substance crystallizes in different crystal packing arrangements all of which have the same elemental composition. Hydrates exist when the drug substance incorporates water in the crystal lattice. Desolvated solvates are produced when a solvate is desolvated and the crystal retains the structure of the solvate. Amorphous forms exist when a solid with no long range order and thus no crystallinity is produced.

Solutions, Emulsions, Suspensions, and Extracts. With regard to solutions, emulsions, suspensions, and extracts, the dosage forms are prepared by employing pharmaceutically and therapeutically acceptable vehicles. The active ingredient(s) may be dissolved in aqueous media, organic solvent or combination of the two, by suspending the drug (if it is insoluble) in an appropriate medium, or by incorporating the medicinal agent into one of the phases of an oil and water emulsion. These dosage forms can be formulated for different routes of administration: orally, introduction into body cavities, or external application.

A solution is a homogeneous mixture that is prepared by dissolving a solid, liquid, or gas in another liquid and represents a group of preparations in which the molecules of the solute or dissolved substance are dispersed among those of the solvent. An emulsion is a two-phase system prepared by combining two immiscible liquids, in which small globules of one liquid are dispersed uniformly throughout the other liquid. The word “suspension” is defined as a two-phase system consisting of an undissolved or immiscible material dispersed in a vehicle (solid, liquid, or gas). Extraction, as the term is used pharmaceutically, involves the separation of medicinally active portions of plant or animal tissues from the inactive or inert components by using selective solvents in standard extraction procedures.

Formulation may influence the bioavailability and pharmacokinetics of drugs in solution, including drug concentration, volume of liquid administered, pH, ionic strength, buffer capacity, surface tension, specific gravity, viscosity and excipients. Emulsions and suspensions are more complex systems. Consequently, the bioavailability and pharmacokinetics of these systems may be affected by additional formulation factors such as surfactants, type of viscosity agent, particle size and particle-size distribution, polymorphism and solubility of drug in the oil phase.

Parenteral Preparations. With respect to parenteral preparations, parenteral dosage forms differ from all other drug dosage forms because they are injected directly into body tissue through the primary protective system of the human body, the skin, and mucous membranes. They must be exceptionally pure and free from physical, chemical, and biological contaminants. These requirements place a heavy responsibility on the pharmaceutical industry to practice current good manufacturing practices (cGMPs) in the manufacture of parenteral dosage forms and upon pharmacists and other health care professionals to practice good aseptic practices (GAPs) in dispensing them for administration to patients.

Certain pharmaceutical agents, particularly peptides, proteins, and antibodies, can only be given parenterally because they are inactivated in the gastrointestinal tract when given by mouth. Parenterally administered drugs are relatively unstable and generally highly potent drugs that require strict control of their administration to the patient. Because of the advent of biotechnology, parenteral products have grown in number and usage around the world.

Formulation principles require that parenteral drugs be formulated as solutions, suspensions, emulsions, liposomes, microspheres, nanosystems, and powders to be reconstituted as solutions. Since most liquid injections are quite dilute, the component present in the highest proportion is the vehicle. The vehicle for most parenteral products is water. The United States Pharmacopeia (USP) requires Water for Injection (WFI). Water-miscible vehicles have been used. Non-aqueous vehicles are another alternative, the most important group being fixed oils. The USP permits substances to be added to a preparation to improve or safeguard its quality, for example, antimicrobial agents, buffers, antioxidants, tonicity agents, and cryoprotectants and lyoprotectants.

Drug pharmacokinetics, solubility, stability, and compatibility with additives dictate the choice of the final formulation of a parenteral drug. So do routes of administration. Injections may be administered by routes such as intravenous, subcutaneous, intradermal, intramuscular, intraarticular, and intrathecal. The type of dosage form (solution, suspension, etc.) will determine the particular route of administration that may be employed. Conversely, the desired route of administration will place requirements on the formulation.

In the preparation of a parenteral product, the general manufacturing process entails procurement, processing, packaging, and QA/QC. Procurement encompasses selecting and testing of the raw-material ingredients and containers. Processing includes cleaning the equipment, compounding the solution (or other dosage form), filtering the solution, sterilizing the containers, filling measured quantities of product into sterile containers, stoppering, freeze-drying, terminal sterilization, and sealing of the filled container. Packaging constitutes the labeling and cartoning of filled and sealed containers. The quality assurance and control unit is responsible for assuring and controlling the quality of the product through the process.

Oral Solid Dosage Forms. Drug substances most frequently are administered orally by means of solid dosage forms such as tablets and capsules, although powders can also be administered as the simplest dosage form. Large-scale production methods used for the preparation of tablets and capsules usually require the presence of other materials in addition to the active ingredients. Additives also may be included in the formulations to facilitate handling, enhance the physical appearance, improve stability, and aid in the delivery of the drug to the bloodstream after administration.

Tablets may be defined as solid pharmaceutical dosage forms containing drug substances with or without suitable diluents and have been traditionally prepared by either compression, or molding methods. Recently, punching of laminated sheets, electronic deposition methods, and three-dimensional printing methods have been used to make tablets.

Compressed tablets are formed by compression and in their simplest form, contain no special coating. They are made from powdered, crystalline, or granular materials, alone or in combination with binders, disintegrants, controlled-release polymers, lubricants, diluents, and in many cases colorants. The vast majority of tablets commercialized today are compressed tablets, either in an uncoated or coated state.

In addition to the active or therapeutic ingredient, tablets contain a number of inert materials. The latter are known as additives or excipients. They may be classified according to the part they play in the finished tablet. The first group contains those that help to impart satisfactory processing and compression characteristics to the formulation. These include diluents (e.g., dicalcium phosphate, calcium sulfate, lactose, cellulose, kaolin, mannitol, sodium chloride, dry starch, and powdered sugar), binders (e.g., starch, gelatin, sugars, gums, cellulosics, and polyvinylpyrrolidone), glidants (e.g., colloidal silicon dioxide), and lubricants (e.g., talc, magnesium stearate, calcium stearate, stearic acid, glyceryl behanate, hydrogenated vegetable oils, and polyethylene glycol). The second group of added substances helps to give additional desirable physical characteristics to the finished tablet. Included in this group are disintegrants (e.g., starches, clays, celluloses, algins, gums, and cross-linked polymers), surfactants, colors, and, in the case of chewable tablets, flavors, and sweetening agents, and in the case of controlled-release tablets, polymers or hydrophobic materials, such as waxes or other solubility-retarding materials. In some cases, antioxidants or other materials can be added to improve stability and shelf-life.

Capsules are solid dosage forms in which the drug substance is enclosed in either a hard or soft, soluble shell of a suitable form of gelatin. Compared with tablets, powders for filling into hard gelatin capsules require a minimum of formulation efforts. The powders usually contain diluents such as lactose, mannitol, calcium carbonate, or magnesium carbonate. Lubricants such as the stearates also are used frequently. The gelatin for soft shell capsules is plasticized typically by the addition of glycerin, sorbitol, or a similar polyol.

Controlled Drug Delivery. Controlled drug delivery can be defined as delivery of the drug at a predetermined rate and/or to a location according to the needs of the body and disease states for a definite time period. In rate-controlled release systems, the mechanism by which the release rate is controlled is diffusion, dissolution, osmosis, mechanically driven pump, swelling, erosion, and stimulation. In targeted delivery systems, targeting is achieved by colloidal drug carriers, ligand-mediated targeting, resealed erythrocytes, bioadhesives, and prodrugs. Device implantation, encapsulated cells, and reservoir microchips are new delivery systems being developed.

Currently, most modified-release delivery systems fall into the following three categories: Delayed-release, extended-release, and site-specific and receptor targeting. Delayed-release systems are either those that use repetitive, intermittent dosing of a drug from one or more immediate-release units incorporated into a single dosage form, or an enteric delayed release system. Extended-release systems include any dosage form that maintains therapeutic blood or tissue levels of the drug for a prolonged period. Site-specific and receptor targeting refers to targeting a drug directly to a certain biological location, a site in the former case, a receptor in the latter case. Recently, a novel modification of drug delivery systems has emerged from the pharmaceutical industry, in which a fast-dissolve drug delivery system consists of a solid dosage form that dissolves or disintegrates in the oral cavity without the need of water or chewing.

Aerosols. Inhalation therapy has been used for many years, and there has been a resurgence of interest in delivery of drugs by this route of administration. The number of new drug entities delivered by the inhalation route has increased over the past 5 to 10 years. This type of therapy also has been applied to delivery of drugs through the nasal mucosa, as well as through the oral cavity for buccal absorption. Originally, this type of therapy was used primarily to administer drugs directly to the respiratory system (treatment of asthma); inhalation therapy is now being used for drugs to be delivered to the bloodstream and finally to the desired site of action. Drugs administered via the respiratory system (inhalation therapy) can be delivered either orally or nasally. Further, these products can be developed as a nebulizer/atomizer, dry powder inhaler, nasal inhaler, or metered dose aerosol inhaler.

Biotechnology Drugs. Recombinant human proteins have been made possible through the biotechnology techniques that allow the production of normally minute amounts of proteins, particularly by use of the DNA that encodes the protein. In antisense RNA and DNA, a short oligonucleotide (10-20 base pairs) complementary to a specific mRNA binds to its target mRNA, which inhibits protein translation by interfering with ribosomal function; additionally, the resulting DNA-RNA duplex recruits the activity of RNase H, a ubiquitous enzyme that degrades the RNA itself. With ribozymes, RNAs possess an enzymatic RNA-degrading activity and are directed toward a specific RNA by the sequence similarity used by antisense molecules. Aptamers are RNA molecules specifically selected by virtue of their three-dimensional nature for high affinity to certain molecular targets. Small interfering RNAs are small RNA molecules that interfere with expression of genes by a mechanism where a type III RNase enzyme (called “Dicer”) is activated to cleave long RNA molecules into 21-28 base pair fragments which then hybridize to other copies of long RNA molecules to catalyze their degradation. In gene therapy, a gene is introduced into the body to help fight a disease. In general, the DNA encoding this gene is encoded on a plasmid molecule or is part of a viral vector that can infect cells with the appropriate desirable gene without causing viral disease. Delivery methods for these gene sources usually either exploit the DNA delivery tactic of the virus itself or employ cationic liposomal complexes with the DNA to mask the plasmid's negative charge.

New Drug Process. New drug development can proceed along varied pathways for different compounds, but a development paradigm has been articulated that has long served well as a general model. In outline form, the paradigm portrays new drug discovery and development as proceeding in a sequence of (possibly overlapping) phases. Discovery programs result in the synthesis of compounds that are tested in preclinical tests called assays and animal models. Clinical (human) testing typically proceeds through three successive phases. In phase I, a small number of usually healthy volunteers are tested to establish safe dosages and to gather information on the absorption, distribution, metabolic effects, excretion, and toxicity of the compound. To conduct clinical testing in the United States, a manufacturer must first file an investigational new drug application (IND) with the Food and Drug Administration (FDA). Phase II trials are conducted with subjects who have the targeted disease or condition and are designed to obtain evidence on safety and preliminary data on efficacy. The number of subjects tested in this phase is larger than in phase I and may number in the hundreds. The final pre-approval clinical testing phase, phase III, typically consists of a number of large-scale (often multi-center) trials that are designed to firmly establish efficacy and to uncover side-effects that occur infrequently. The number of subjects in phase III trials for a compound can total in the thousands. Once drug developers believe that they have enough evidence of safety and efficacy, they will compile the results of their testing in an application to regulatory authorities for marketing approval. In the United States, manufacturers submit a new drug application (NDA) or a biological license application (BLA) to the FDA for review and approval.

Dosages. The dose of a drug required to produce a specified effect in 50% of the population is the median effective dose, abbreviated ED₅₀. In preclinical studies of drugs, the median lethal dose, as determined in experimental animals, is abbreviated as the LD₅₀. The ratio of the LD₅₀ to the ED₅₀ is an indication of the therapeutic index, which is a statement of how selective the drug is in producing the desired versus its adverse effects. Drugs that exhibit high therapeutic indices are preferred.

What is claimed is:

1. A method for treating or preventing diffuse parenchymal lung disease in an individual in need thereof comprising:
administering a pharmaceutical composition comprising a selective active-site mTOR inhibitor to said individual in a therapeutically effective amount to treat or prevent pulmonary fibrosis in said individual.

2. A method for determining therapeutic efficacy of treatment with an active-site mTOR inhibitor in an individual in need thereof comprising:
a. Administering a pharmaceutical composition comprising a selective active-site mTOR inhibitor to said individual in a therapeutically effective amount to treat or prevent pulmonary fibrosis in said individual; and
b. measuring lung function to assess therapeutic efficacy wherein improved lung function indicates therapeutic efficacy.

3. A method for inhibiting expression of α-SMA or collagen or fibronectin in a pulmonary fibroblast comprising administering an amount of a selective active-site mTOR inhibitor to a pulmonary fibroblast in an individual suffering from a diffuse parenchymal lung disease effective to inhibit expression of α-SMA or collagen or fibronectin in said pulmonary fibroblast of said patient.

4. The method of claim 1, 2, or 3, wherein said diffuse parenchymal lung disease (DPLD) is a DPLD of environmental cause.

5. The method of claim 1, 2, or 3, wherein said diffuse parenchymal lung disease is a collagen vascular disease.

6. The method of claim 1, 2, or 3, wherein said diffuse parenchymal lung disease is a idiopathic interstitial pneumonia.

7. The method of claim 1, 2, or 3, wherein said diffuse parenchymal lung disease is sarcoidosis.

8. The method of claim 5, wherein said collagen vascular disease is scleroderma.

9. The method of claim 5, wherein said collagen vascular disease is rheumatoid arthritis.

10. The method of claim 6, wherein said idiopathic interstitial pneumonia is idiopathic pulmonary fibrosis (IPF).

11. The method of claim 6, wherein said idiopathic interstitial pneumonia is nonspecific interstitial pneumonia (NSIP).

12. The method of claim 1, 2, or 3, wherein said selective active-site mTOR inhibitor is a pyrazolopyrimidine.

13. The method of claim 1, 2, or 3, wherein said selective active-site mTOR inhibitor is defined as being selective on the basis of inhibiting other PI3Ks only at ≧ about 10-fold higher concentrations.

14. The method of claim 1, 2, or 3, wherein said selective active-site mTOR inhibitor is defined as being an ATP-competitive inhibitor of mTOR on the basis of one or more assays reported in Thoreen C. C. et al., J. Biol. Chem., 2009, 284, 8023-8032; Feldman M. E. et al., PLoS Biol., 2009, 7, e38; García-Martínez J. M. et al., Biochem. J., 2009, 421, 29-42; or Yu K. et al., Cancer Res., 2009, 69, 6232-6240.

15. The method of claim 14, wherein said selective active-site mTOR inhibitor is Torin 1.

16. The method of claim 14, wherein said selective active-site mTOR inhibitor is PP242.

17. The method of claim 14, wherein said selective active-site mTOR inhibitor is Ku-0063794.

18. The method of claim 14, wherein said selective active-site mTOR inhibitor is WAY-600.
